(19) United States
(12) Patent Application Publication
(10) Pub. No.: US 2023/0218539 A1
(43) Pub. Date: Jul. 13, 2023

(54) TRANSDERMAL THERAPEUTIC SYSTEM COMPRISING THE INGREDIENT ROTIGOTINE AND AT LEAST ONE NON-AMINE-RESISTANT SILICONE ADHESIVE

(71) Applicant: Luye Pharma Switzerland AG, Basel (CH)

(72) Inventors: Christian BENDA, Holzkirchen (DE); Ulrike KOLLING, Rosenheim (DE); Bjoern SCHURAD, Munich (DE); Gabriel WAUER, Schliersee (DE)

(73) Assignee: Luye Pharma Switzerland AG, Basel (CH)

(21) Appl. No.: 17/794,886

(22) PCT Filed: Jan. 22, 2021
(86) PCT No.: PCT/EP2021/051500

§ 371 Date: Mar. 29, 2023

(30) Foreign Application Priority Data
Jan. 24, 2020 (EP) 20153621.6

(51) Int. Cl. A61K 9/70; A61K 31/381; A61K 47/32

(52) U.S. Cl. CPC A61K 9/7084; A61K 31/381; A61K 47/32

(57) ABSTRACT

The invention relates to a transdermal therapeutic system (TTS) for the administration of the active ingredient rotigotine comprising a matrix layer containing rotigotine, one or more non-amine-resistant silicone adhesives in an amount of more than 50% by weight, based on the total weight of the pressure sensitive adhesive of the matrix layer, and paraffin, as well as methods for the preparation thereof. The transdermal therapeutic system according to the invention is particularly suitable for the treatment of the Parkinson disease.

### PRIORITY

This application corresponds to the U.S. National phase of International Application No. PCT/EP2021/051500, filed Jan. 22, 2021, which, in turn, claims priority to European Patent Application No. 20153621.6 filed Jan. 24, 2020, the contents of which are incorporated by reference herein in their entirety.

### FIELD OF THE INVENTION

The present invention relates to a transdermal therapeutic system (TTS) for the administration of the active ingredient rotigotine comprising one or more non-amine-resistant silicone adhesives as well as methods for the preparation thereof. The transdermal therapeutic system according to the invention is particularly suitable for the treatment of the Parkinson disease.

### BACKGROUND OF THE INVENTION

Rotigotine is the INN of compound (+5,6,7,8-tetrahydro-6-[propyl-[2-(2-thienyl)ethyl]-amino]-1-naphthalenol of the following structural formula:

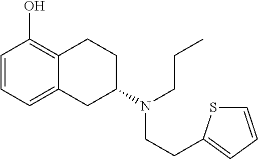 [SMILES: CCCN(CCc1cccs1)[C@H]1CCc2c(O)cccc2C1]

At present, two crystalline forms of rotigotine are known: the polymorphic form I and the polymorphic form II (WO 2009/068520). Polymorphic form I and polymorphic form II can be distinguished by their respective physical-chemical parameters such as powder x-ray diffraction diffractogram, Raman spectrum and melting point. As described in WO 2009/068520 the polymorphic form II is thermodynamically more stable than the polymorphic form I and is also said to have improved processing properties.

Rotigotine is a known dopamine receptor agonist that is successfully employed for the treatment of the Parkinson disease. Pharmaceutical efficacy of rotigotine and diseases in which rotigotine can preferably be used are described for example in WO 2002/089777, WO 2005/092331, WO 2005/009424, WO 2003/092677, and WO 2005/063237.

Oral administration of the compound rotigotine is complicated due to its short half-life and its high First-Pass-Effect. Therefore, a number of printed matters suggest transdermal administration of rotigotine and a corresponding drug named Neupro® is on the market.

A transdermal therapeutic system for the administration of rotigotine has already early been described in WO 94/07468. In the system described there the active ingredient is used as hydrochloride in a two-phase matrix which is substantially formed by a hydrophobic polymer material present as a continuous phase with hydrated silicate dispersed therein for taking up the hydrophilic drug salt. However, the transdermal therapeutic systems described there are difficult to prepare and skin permeation of the active ingredient from said system is complicated. Therefore, the transdermal therapeutic systems described in WO 94/07468 were not marketable.

There are a number of printed matters which describe improved transdermal therapeutic systems. So, WO 99/49852 discloses transdermal therapeutic systems having a matrix based on a non-aqueous polymer adhesive system on an acrylate or silicone basis, respectively, which is substantially free from inorganic silicate particles. The matrix systems in their simplest embodiment represent one-phase matrix systems. The matrix substantially consists of an acrylate adhesive or a silicone adhesive and in the case of a silicone adhesive the matrix can also contain e.g., polyvinylpyrrolidone, copolymers of vinylpyrrolidone and vinyl acetate, polyethylene glycol, glycerin, fatty acid esters of glycerin, or copolymers of ethylene and vinyl acetate.

WO 2004/012730, WO 2004/012719, and WO 2004/058247 disclose transdermal therapeutic systems for the administration of rotigotine having a self-adherent matrix which is saturated with the active ingredient and contains the active ingredient as a number of micro-reservoirs or amorphous particles, respectively. The matrix is a self-adherent matrix based on a silicone adhesive.

A transdermal therapeutic system for the administration of rotigotine based on a silicone matrix is also disclosed in WO 02/089778. Here, it is regarded to be essential that the transdermal therapeutic system has a surface area of 10-40 cm² and contains 0.1-3.15 mg/cm² of rotigotine as the active ingredient.

An important aspect in the formulation of transdermal therapeutic systems with the active ingredient rotigotine is to avoid the crystallization of the active ingredient in the transdermal therapeutic system. Crystallized active ingredient can cause a number of problems, on the one hand administration rate is impaired, on the other hand also the adhesive properties of the adhesive matrix can adversely be affected. Unfortunately, such crystallization is presented as a common problem for rotigotine in the prior art. In the only transdermal therapeutic system that is on the market at present, the product “Neupro®” by UCB, rotigotine is present embedded in a polymer matrix. However, a first formulation has not proved to be stable and resulted in the formation of crystals in the product which is why a recall action had to be carried out.

Hence, WO 2012/072650 suggests to insert the active ingredient into a non-adherent matrix and to provide an adhesive layer on the non-adherent matrix which preferably is self-adherent and preferably consists of a “pressure sensitive adhesive”, preferably of an amine-resistant silicone adhesive.

WO 2012/084969 suggests transdermal therapeutic systems for the administration of rotigotine in which the adhesive matrix consists of polystyrene, polyisobutylene and mixtures thereof and in addition to the active ingredient also contains at least one cross-linked polyvinyl pyrrolidone or a copolymer of vinyl pyrrolidone and vinyl acetate.

Printed matter WO 2011/076879 describes that polyvinyl pyrrolidone has to be used in a certain weight ratio to rotigotine, since such a weight ratio unexpectedly would be able to stabilize the non-crystalline form of rotigotine and to prevent rotigotine from recrystallizing in a solid dispersion, such as a self-adherent matrix of a transdermal therapeutic system. Therefore, WO 2011/076879 suggests to use polyvinyl pyrrolidone for stabilizing a solid dispersion of the non-crystalline form of rotigotine in a dispersing agent, which preferably comprises at least one pressure sensitive silicone adhesive, in a weight ratio of rotigotine to polyvinyl pyrrolidone from about 9:3.5 to about 9:6.

Finally, printed matter WO 2011/057714 describes a method for preventing crystallization of a drug in a polymer film. Said polymer films of WO 2011/057714 among others are suitable to prepare transdermal therapeutic systems, wherein one of the two preferred drugs is rotigotine. Here, the solvent-containing coating mass, which was spread out in the preparation of the polymer film and comprises a matrix-forming polymer or polymer mixture and at least one drug, has to be dried at temperatures which occasionally are at least 10° C. above the melting temperature of the drug contained in the coating mass.

A further important aspect in the formulation of transdermal therapeutic systems is the choice of the pressure sensitive adhesive. When choosing the pressure sensitive adhesive various factors have to be considered, in particular an optimum adhesion strength, separation force and tack, constant release of the active ingredient, stability in storage, as well as minimal adhesive residues and skin irritations shall be achieved. In addition, the pressure sensitive adhesives shall not react with the active ingredient.

“Adhesiveness” (or “adhesive strength”) describes the force required to peel off a transdermal therapeutic system from a test surface to which the transdermal therapeutic system has been attached, i.e. the property to withstand the peeling off from a surface. “Separation force” describes the force required to peel off a TTS from the release liner (4). “Tack” is the property to bond to solid surfaces, i.e. the property to adhere to a solid surface with a short contact time and very slight pressure.

Various pressure sensitive adhesives for use in transdermal therapeutic systems with the active ingredient rotigotine have been suggested in the prior art, in particular silicone adhesives, but for example also polyacrylate, polyisobutylene, and polystyrene based polymer adhesives.

Polyisobutylene and polystyrene and mixtures thereof for example are used in the transdermal therapeutic systems for the administration of rotigotine of WO 2012/084969. Polyisobutylenes basically have poor solution properties compared to many pharmaceutical active ingredients and in addition are disadvantageous in that they possess sufficient tack only in a mixture with lower molecular polyisobutylenes and then, can also possess a higher so-called cold flow. Meanwhile, styrenes generally need high amounts of plasticizers and tackifiers.

On the other hand, pressure sensitive silicone adhesives have a high flexibility, low surface tension, and minimal changes in properties over a wide temperature range. Finally, silicone adhesives have a high air and water permeability, good cutaneous tolerance, and resistance over outside influences (moisture, ultraviolet rays, stability under acidic and basic conditions, etc.).

In case of pressure sensitive silicone adhesives for transdermal therapeutic systems a basic distinction can be made between two types, namely “non-amine-resistant” silicone adhesives and “amine-resistant” silicone adhesives. “Non-amine-resistant” silicone adhesives additionally contain free silanol groups. Said silanol groups slightly interact with the amine groups in active ingredients such as rotigotine, whereby active ingredient degradation products can occur. Moreover, said reaction can significantly impair the properties of the adhesive layer in transdermal therapeutic systems, for example by reducing the tack and/or by drying up during storage (see e.g., U.S. printed patent specification U.S. RE35,474 and U.S. Pat. No. 4,591,622).

For this reason, in the prior art for silicone adhesive-based transdermal therapeutic systems with the active ingredient rotigotine in general only an amine-resistant and no non-amine-resistant silicone adhesive is used. In an “amine-resistant” silicone adhesive the silanol groups are protected by protective groups, for example by trimethylsilyl, abbreviated (TMS) groups.

So, for example in the only transdermal therapeutic system Neupro® that is available on the market at present exclusively amine-resistant silicone adhesives are used. This is a single-layer laminate with the matrix layer as a biphasic system in the form of a solid dispersion, wherein an inner phase dissolved by the active ingredient in a polymer and an outer phase by an amine-resistant silicone adhesive as a dispersing agent are formed.

In the same way, in WO 99/49852 due to the basic nature of rotigotine, amine-resistant adhesives are employed for a silicone adhesive containing said active ingredient. As described in WO 99/49852 such amine-resistant silicone adhesives are characterized in that they have no free silanol functions (i.e., silanol groups).

According to WO 02/089778 also the transdermal therapeutic system on the basis of silicone disclosed therein has to contain at least one amine-resistant silicone compound as the main component. Here, the silicone compound usually is a pressure sensitive adhesive or a mixture thereof and forms a matrix in which the other components of the transdermal therapeutic system are embedded.

According to WO 2004/012730 (and also according to WO 2004/012719 and WO 2011/076879) particularly preferred pressure sensitive adhesives for use in the transdermal therapeutic systems disclosed therein are of the type forming a network of soluble poly-condensed polydimethylsiloxane (PDMS)/resin, wherein the hydroxy groups are protected e.g. with trimethylsilyl (TMS) groups.

According to WO 2004/058247 in a preferred embodiment of the invention the matrix polymer is a silicone, preferably an amine-resistant silicone or a silicone mixture. In the same way, according to WO 2004/058247 the matrix polymer is an amine-resistant silicone or a mixture of amine-resistant silicones. Finally, also according to WO 2011/057714 amine-resistant polysiloxanes are particularly preferred.

However, such amine-resistant silicone adhesives, such as for example used in the marketed product Neupro®, often lead to unsatisfactory results, particularly in view of adhesion strength and tack.

Thus, despite all the known transdermal therapeutic systems with the active ingredient rotigotine there is a need for a transdermal therapeutic system for the administration of the active ingredient rotigotine which has a satisfactory adhesion strength and tack and an excellent stability in storage, i.e., in which the active ingredient does not crystallize during the shelf life. The transdermal therapeutic system nevertheless shall ensure a sufficient skin permeation for the active ingredient, to be manufactured as simple as possible and at the same time allow administration of the active ingredient over the desired duration of administration of at least one day. Further, for reasons of costs and in view of requirements of certain national drug regulatory authorities (e.g., to avoid abuse) the residual content of the active ingredient in the transdermal therapeutic system should not be too high after having been used.

### SUMMARY OF THE INVENTION

To solve this problem the invention suggests transdermal therapeutic systems as defined in the claims.

The authors of the present invention have surprisingly found that in transdermal therapeutic systems (TTS) with the active ingredient rotigotine by the addition of a small amount of paraffin to the matrix layer improved properties, in particular a significantly increased adhesion strength and tack and improved stability in storage can be achieved despite the use of silicone adhesives still having a relevant amount of free silanol groups (i.e., non-amine-resistant silicone adhesives) compared to a TTS in which amine-resistant silicone adhesives are used.

Moreover, the present inventors have found that in such a transdermal therapeutic system with the active ingredient rotigotine and one or more non-amine-resistant silicone adhesives in an amount of more than 50% by weight, based on the total weight of the pressure sensitive adhesive of the matrix layer (2), when using rotigotine and a polyvinyl pyrrolidone in the matrix layer in the dispersed phase of a solid dispersion in a weight ratio of 9:6.4, particularly 9:7 or smaller there is no crystallization of the active ingredient. On the other hand, with a greater weight ratio of rotigotine and polyvinyl pyrrolidone, for example of 9:5 or greater, there is the risk that after elongated shelf lives, at temperatures of 25° C. or higher, formation of crystals can occur. Hence, such high weight ratios of rotigotine to polyvinyl pyrrolidone according to the invention certainly are possible, but not preferred.

Finally, it has surprisingly been found that a drying temperature in the coating operation to at least 10° C. above the melting point of rotigotine to prevent a later crystallization of rotigotine is not required in the transdermal therapeutic systems according to the invention.

As a result, the invention provides a transdermal therapeutic system comprising a backing layer (1), a matrix layer (2) containing a drug and a release liner (4) to be removed before use, wherein the drug is rotigotine and wherein the matrix layer (2) contains one or more non-amine-resistant pressure sensitive silicone adhesives in an amount of more than 50% by weight, based on the total weight of the pressure sensitive adhesive of the matrix layer (2), and paraffin in an amount of at least 0.1% by weight, based on the total weight of the matrix layer (2).

The invention also relates to the use of the transdermal therapeutic systems for the treatment of diseases in which a transdermal administration of rotigotine is indicated, in particular for the treatment of the Parkinson disease. Finally, the invention relates to methods for the preparation of the transdermal therapeutic systems according to the invention.

### BRIEF DESCRIPTION OF THE DRAWINGS

FIG. 1: (A) Monolayer formulation of the transdermal therapeutic system having a backing layer (1), a matrix layer (2) and a release liner (4). (B) Bilayer formulation of the transdermal therapeutic system having a backing layer (1), a matrix layer (2), at least one additional initially active ingredient-free pressure sensitive adhesive layer (3) and a release liner (4).

FIG. 2: Separation force of various monolayer and bilayer formulations during storage for 0 to 3 months at 40° C./75% rh (relative humidity).

FIG. 3: Adhesion strength of various monolayer and bilayer formulations during storage for 0 to 3 months at 40° C./75% rh (relative humidity).

FIG. 4: (A) Determination of the optimum polymer adhesive ratio of the silanol-reduced silicone adhesives BIO-PSA SRS7-4501 (medium tack): SRS7-4601 (high tack) considering separation force, adhesion strength and tack. (B) Determination of the optimum polymer adhesive ratio of the non-silanol-reduced silicone adhesives BIO-PSA 7-4501 (medium tack): 7-4601 (high tack) considering separation force, adhesion strength and tack.

FIG. 5: Tack of various monolayer and bilayer formulations during storage for 0 to 3 months at 40° C./75% rh (relative humidity).

FIG. 6: A) Cumulatively permeated amount of rotigotine over 24 hrs in monolayer formulations with a constant content of rotigotine and a variable content of PVP K90. B) Cumulatively permeated amount of rotigotine over 24 hrs of monolayer formulations with different mixing ratios of BIO-PSA SRS7-4501 to BIO-PSA SRS7-4601 (Dow Corning®) at constant rotigotine and PVP K90 contents.

FIG. 7: (A) Cumulatively permeated amount of rotigotine over 24 hrs in a monolayer formulation with silanol-reduced non-amine-resistant silicone adhesive and 2% by weight of paraffin. (B) Cumulatively permeated amount of rotigotine over 24 hrs in monolayer formulations with non-silanol-reduced non-amine-resistant silicone adhesives with an amount of paraffin of from 1-2% by weight.

FIG. 8: A) Cumulatively permeated amount of rotigotine over 24 hrs in bilayer formulations. B) Cumulatively released amount of rotigotine over 6 hrs in monolayer formulations.

FIG. 9 Cumulatively released amount of rotigotine over 6 hrs in a bilayer formulation per in vitro dissolution (in vitro release of active ingredient) in which the mass of the active ingredient-containing matrix layer (2) before the respective coating with ca. 50 g/m² each was only stirred and the same mass before the respective coating with 50 g/m² was additionally homogenized. 618_617 ROTTDS: Bilayer formulation; 589ROTTDS: Monolayer formulation; stirred: only stirred; homogenized: stirred and homogenized.

### DETAILED DESCRIPTION OF THE INVENTION

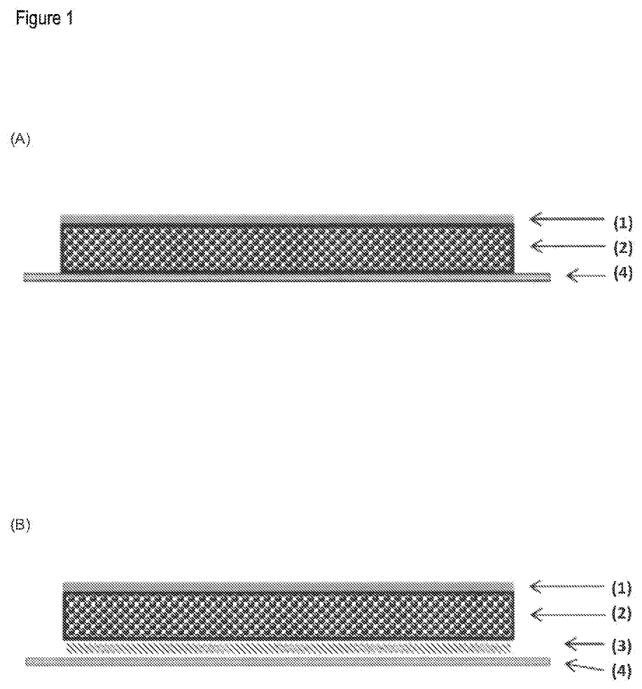

In its simplest design the transdermal therapeutic system according to the invention comprises a backing layer (1), the backing layer (1) is followed by a matrix layer (2) containing the active ingredient and the matrix layer (2) is followed by a release liner (4) to be removed before use (cf., FIG. 1 (A)). Between the individual layers of the transdermal therapeutic system according to the invention, i.e., between the backing layer (1) and the matrix layer (2) and/or between the matrix layer (2) and the release liner (4) one or more further layers may be present each.

For example, in a preferred embodiment of the present invention at least one additional initially active ingredient-free pressure sensitive adhesive layer (3) may be present between the matrix layer (2) and the release liner (4).

The matrix layer (2) of the transdermal therapeutic system according to the invention is a pressure sensitive adhesive layer containing the active ingredient rotigotine and according to the invention it is essential that said pressure sensitive adhesive layer contains one or more non-amine-resistant pressure sensitive silicone adhesives in an amount of more than 50% by weight, based on the total weight of the pressure sensitive adhesive of the matrix layer (2), and paraffin in an amount of at least 0.1% by weight, based on the total weight of the matrix layer (2).

The present inventors have surprisingly found that, by adding a small amount of paraffin to the matrix layer (2) a significant increase in the adhesion strength and tack and an improved stability in storage, in particular regarding the adhesion strength and tack can be achieved, despite at the same time using non-amine-resistant silicone adhesives and the active ingredient rotigotine in the matrix layer (2) compared to a transdermal therapeutic system in which amine-resistant silicone adhesives and rotigotine are used.

As used herein, the terms “total weight” and “total amount” each relate to the dry weight, i.e., the weight of the constituents to which the terms “total weight” or “total amount” relate in the corresponding context in the transdermal therapeutic system in its ready-to-use form, unless otherwise obvious.

The “pressure sensitive adhesives” of a transdermal therapeutic system or the “pressure sensitive adhesives” of a layer of a transdermal therapeutic system, for example the matrix layer (2) and/or the at least one additional initially active ingredient-free pressure sensitive adhesive layer (3), in the sense of the present invention are meant to be all those constituents, in particular polymer adhesives, that are added to the transdermal therapeutic system or to the layer(s) of the transdermal therapeutic system due to their inherent property as a pressure sensitive adhesion promoter/adhesive. Such pressure sensitive adhesives are known to the skilled person. Thus, the active ingredient, paraffin, a crystallization inhibitor such as polyvinyl pyrrolidone, penetration enhancers and further additives such as plasticizers and antioxidants are not among the pressure sensitive adhesives. As used herein, the terms “silicone adhesive” and “pressure sensitive silicone adhesive” are interchangeable.

The matrix layer (2) of the transdermal therapeutic system according to the invention comprises one or more non-amine-resistant silicone adhesives in an amount of more than 50% by weight based on the total weight of the pressure sensitive adhesive of the matrix layer (2). That is, the matrix layer (2) can comprise one, two, three, four etc. non-amine-resistant silicone adhesives.

The skilled person knows what the terms “non-amine-resistant” silicone adhesive and “amine-resistant” silicone adhesive are meant to be. Amine-resistant silicone adhesives and methods for the preparation of such amine-resistant silicone adhesives, respectively are described for example in the U.S. printed patent specifications U.S. RE35,474 and U.S. Pat. No. 4,591,622. For example, a “non-amine-resistant” silicone adhesive is characterized in that it is a pressure sensitive silicone adhesive which unlike an “amine-resistant” silicone adhesive has a relevant amount of free silanol groups (OH groups not protected by protective groups), so that under ordinary circumstances there is the risk of an interaction with amine-containing active ingredients. Uncomplete protection, so-called capping or (end-) blocking or only partial removal of said free silanol groups results in the so-called silanol-reduced silicone adhesives. Silanol-reduced non-amine-resistant silicone adhesives and methods for the preparation of such silanol-reduced non-amine-resistant silicone adhesives are known to the skilled person and described for example in the U.S. printed patent specification U.S. Pat. No. 6,337,086. They still belong to the group of the non-amine-resistant silicone adhesives.

Preferably, the term “non-amine-resistant” silicone adhesive in the sense of the present invention is meant to be a pressure sensitive silicone adhesive having a content of free silanol groups (content of free OH groups or silicium-bound hydroxyl content) of for example at least 7700 ppm or more, preferably at least about 8000 ppm or more, and preferably not more than 13000 ppm. The content (or concentration) of free silanol groups in silicone adhesives can be measured by means of methods familiar to the skilled person such as for example by means of nuclear magnetic resonance spectroscopy (NMR spectroscopy) and/or Fourier transform infrared spectroscopy (FTIR spectroscopy) (see, for example U.S. Pat. No. 6,337,086). Here, the silanol content can be determined by means of ²⁹Si nuclear magnetic resonance spectroscopy (²⁹Si-NMR spectroscopy) and correlation of the data against standardized reference samples, while the FTIR spectroscopy directly provides a ratio of free to protected silanol functions.

So, for example as described in U.S. Pat. No. 6,337,086, the silanol content can be calculated by means of FTIR spectroscopy via the peak area ratio A1/(A2*100), wherein the A1 area corresponds to the peak for the dimeric dilatation mode of the O—H bond from the silanol groups and the A2 area corresponds to the area for an overtone peak of the deformation of the hydrogen of the polydimethylsiloxane (PDMS) methyl group. In this embodiment a non-amine-resistant silicone adhesive is characterized in that its ratio of unprotected to protected silanol functions for example is greater than 0.45, preferably at least 0.46, more preferably at least 0.5 or more.

In a preferred embodiment of the present invention a non-amine-resistant silicone adhesive is characterized in that after it has been reacted with the active ingredient rotigotine at ca. 50° C. for at least two hours, preferably two to four hours, in a suitable solvent and in a mixing ratio of silicone adhesive to rotigotine of for example 20:1 to 4:1, preferably for example 10:1, a not inconsiderable part of the rotigotine, for example at least 0.5% by weight, preferably at least 1.0% by weight, preferably at least 2.5% by weight, reacts with the silicone adhesive and thereby is degraded or converted, respectively. Suitable solvents are known to the skilled person and particularly depend on the silicone adhesive; examples are heptane, ethanol, and ethyl acetate. The proportion of degraded or converted, respectively rotigotine can be determined in a manner known to the skilled person.

In view of the choice of the one or more non-amine-resistant silicone adhesives otherwise the invention is not particularly limited. Non-amine-resistant silicone adhesives are known from the prior art.

In one embodiment of the invention the one or more non-amine-resistant pressure sensitive silicone adhesives have to be selected from the group comprising non-amine-resistant pressure sensitive silicone adhesives of medium tack, such as for example Dow Corning® BIO-PSA 7-4501, Dow Corning® BIO-PSA 7-4502, Dow Corning® BIO-PSA SRS7-4501, Dow Corning® BIO-PSA SRS7-4502, non-amine-resistant pressure sensitive silicone adhesives of high tack, such as for example Dow Corning® BIO-PSA 7-4601, Dow Corning® BIO-PSA 7-4602, Dow Corning® BIO-PSA SRS7-4601, Dow Corning® BIO-PSA SRS7-4602 and combinations thereof. Chemically these adhesives are also referred to as dimethiconol trimethyl siloxysilicate cross-polymer. In a further embodiment of the invention the one or more non-amine-resistant pressure sensitive silicone adhesives have to be selected from the group comprising non-silanol-reduced non-amine-resistant pressure sensitive silicone adhesives of medium tack, non-silanol-reduced non-amine-resistant pressure sensitive silicone adhesives of high tack, silanol-reduced non-amine-resistant pressure sensitive silicone adhesives of medium tack, silanol-reduced non-amine-resistant pressure sensitive silicone adhesives of high tack and combinations thereof. Examples of preferred non-amine-resistant pressure sensitive silicone adhesives in the sense of the present invention are Dow Corning® BIO-PSA 7-4501, Dow Corning® BIO-PSA 7-4601, Dow Corning® BIO-PSA SRS7-4501, Dow Corning® BIO-PSA SRS7-4601, and combinations thereof.

The matrix layer (2) of the transdermal therapeutic system according to the invention contains one or more non-amine-resistant pressure sensitive silicone adhesives in an amount of more than 50% by weight based on the total weight of the pressure sensitive adhesive of the matrix layer (2). That is, the matrix layer (2) contains a total weight percentage of non-amine-resistant pressure sensitive silicone adhesive of more than 50% by weight, based on the total weight of the pressure sensitive adhesive of the matrix layer (2).

In a preferred embodiment of the present invention the matrix layer (2) of the transdermal therapeutic system contains weight percentages of non-amine-resistant pressure sensitive silicone adhesive of more than 60% by weight, preferably more than 70% by weight, more preferably more than 75% by weight, more preferably more than 80% by weight, more preferably more than 85% by weight, more preferably more than 90% by weight, further preferred more than 93% by weight, further preferred more than 95% by weight, further preferred at least 99% by weight, based on the total weight of the pressure sensitive adhesive of the matrix layer (2). In a further preferred embodiment of the present invention the matrix layer (2) of the transdermal therapeutic system as pressure sensitive adhesive contains only non-amine-resistant pressure sensitive silicone adhesives; i.e., in particular the matrix layer (2) contains no other polymer adhesives, but only non-amine-resistant pressure sensitive silicone adhesives.

When in the pressure sensitive adhesive layer or pressure sensitive adhesive layers of the TTS to which reference is made (for example the matrix layer (2) or an additional initially active ingredient-free pressure sensitive adhesive layer (3)) exactly one non-amine-resistant silicone adhesive is contained, the weight percentage of said non-amine-resistant silicone adhesive, based on the total weight of the pressure sensitive adhesive of the pressure sensitive adhesive layer or pressure sensitive adhesive layers of the TTS referred to, is more than 50% by weight; when in the pressure sensitive adhesive layer or pressure sensitive adhesive layers of the TTS to which reference is made exactly two non-amine-resistant silicone adhesives are contained, the total weight percentage of said two non-amine-resistant silicone adhesives, based on the total weight of the pressure sensitive adhesive of the pressure sensitive adhesive layer or pressure sensitive adhesive layers of the TTS referred to, is more than 50% by weight; etc. Here, in case of several pressure sensitive adhesive layers in each of these layers a weight percentage of more than 50% by weight each based on the total weight of the pressure sensitive adhesive of the respective layer has to be present.

The matrix layer (2) of the transdermal therapeutic system according to the invention contains paraffin, also referred to as white oil, in an amount of at least 0.1% by weight based on the total weight of the matrix layer (2). In particular, two paraffins are known, namely on the one hand viscous paraffin which in the Ph. Eur. is referred to as paraffin, liquid or paraffinum liquidum, in the USP as mineral oil, in JP as liquid paraffin and in common literature also as paraffinum subliquidum and represents an oily liquid with a relative density according to Ph. Eur. in the range of from 0.827 to 0.890 (Method 2.2.5), according to USP in the range of from 0.845 to 0.905 (Method <841>), and according to JP in the range of from 0.860 to 0.890 and a viscosity according to Ph. Eur. in the range of from 110-230 mPas (Method 2.2.9), according to USP in the range of from 34.5 to 150.0 mm²*s⁻¹ (Method <911> capillary viscometer at 40±0.1°) and according to JP in the range of not less than 37 mm²/s (Method 1, 37.8° C.).

On the other hand, there in known thin paraffin which in the Ph. Eur. is referred to as paraffin, light liquid or paraffinum perliquidum, in USP as light mineral oil and in JP as light liquid paraffin and represents oily liquid with a density according to Ph. Eur. in the range of from 0.810 to 0.875 (Method 2.2.5), according to USP in the range of from 0.818 to 0.880 (Method <841>) and according to JP in the range of from 0.830 to 0.870 and a viscosity according to Ph. Eur. in the range of from 25-80 mPas (Method 2.2.9), according to USP in the range of from 3.0 to 34.4 mm²*s⁻¹ (Method <911> capillary viscometer at 40±0.1°) and according to JP in the range of less than 37 mm²/s (Method 1, 37.8° C.).

Viscous paraffin is preferred. In a further embodiment the paraffin is thin paraffin.

The amount of paraffin in the matrix layer (2) of the transdermal therapeutic systems according to the invention e.g., can be up to 50% by weight based on the total weight of the matrix layer (2) of the transdermal therapeutic systems according to the invention, preferably up to 40% by weight, or preferably up to 30% by weight, more preferably up to 20% by weight, more preferably up to 15% by weight, more preferably up to 10% by weight.

In a preferred embodiment of the present invention the paraffin in the matrix layer (2) is in an amount of at least 0.2% by weight, preferably at least 0.3% by weight, more preferred at least 0.5% by weight, more preferably at least 0.8% by weight, more preferably at least 1.0% by weight, based on the total weight of the matrix layer (2). In a further preferred embodiment of the invention the paraffin is present in the matrix layer (2) in an amount of at least 0.1-30.0% by weight, preferably 0.1-20.0% by weight, more preferably 0.2-20.0% by weight, more preferably 0.5-10.0% by weight, more preferably 0.8-5.0% by weight, more preferably 1.0-5.0% by weight, more preferably 1.0-3.0% by weight, based on the total weight of the matrix layer (2).

Since the matrix layer in the transdermal therapeutic systems according to the invention is self-adhesive it is generally not required for an additional self-adhesive layer to be present on the matrix layer (2), such as it is demanded in WO 2012/072650. However, according to the invention it is also not excluded that at least one such additional initially active ingredient-free pressure sensitive adhesive layer (3) is provided, for example to improve the adhesion strength and tack. In a preferred embodiment of the invention the transdermal therapeutic system according to the invention contains no additional initially active ingredient-free pressure sensitive adhesive layer (3) between the matrix layer (2) and the release liner (4) to be removed before use. According to said embodiment, the matrix layer (2) is sufficiently tacky to ensure an advantageous adhesion on the skin for the desired application period.

In a further preferred embodiment of the invention the transdermal therapeutic system according to the invention contains at least one additional initially active ingredient-free pressure sensitive adhesive layer (3) between the matrix layer (2) and the release liner (4) to be removed before use (cf., FIG. 1 B). The at least one additional initially active ingredient-free pressure sensitive adhesive layer (3) especially in case of a matrix layer (2) of several phases can result in a higher robustness over different sphere size distributions of the active ingredient-containing inner phase during in-vitro dissolution, since the diffusion distance from the active ingredient-containing matrix layer (2) towards the skin through the at least one initially active ingredient-free pressure sensitive adhesive layer (3) is brought to a fixed layer thickness.

In a further preferred embodiment the transdermal therapeutic system according to the invention therefore contains at least one additional initially active ingredient-free pressure sensitive adhesive layer (3) between the matrix layer (2) and the release liner (4) to be removed before use, wherein the at least one additional initially active ingredient-free pressure sensitive adhesive layer (3) contains one or more non-amine-resistant pressure sensitive silicone adhesives in an amount of more than 50% by weight, based on the total weight of the pressure sensitive adhesive of the at least one additional initially active ingredient-free pressure sensitive adhesive layer (3), and paraffin in an amount of at least 0.1% by weight, preferably 0.2-20.0% by weight, more preferably 1.0-5.0% by weight, based on the total weight of the at least one additional initially active ingredient-free pressure sensitive adhesive layer (3). That is, the at least one additional initially active ingredient-free pressure sensitive adhesive layer (3) can comprise one, two, three, four etc. non-amine-resistant silicone adhesives.

In a further preferred embodiment of the present invention the at least one additional initially active ingredient-free pressure sensitive adhesive layer (3) of the transdermal therapeutic system has a weight percentage of non-amine-resistant pressure sensitive silicone adhesive of more than 60% by weight, preferably more than 70% by weight, more preferably more than 75% by weight, more preferably more than 80% by weight, more preferably more than 85% by weight, more preferably more than 90% by weight, further preferred more than 93% by weight, further preferred more than 95% by weight, further preferred at least 99% by weight, based on the total weight of the pressure sensitive adhesive of the at least one additional initially active ingredient-free pressure sensitive adhesive layer (3). In a further embodiment of the present invention the at least one additional initially active ingredient-free pressure sensitive adhesive layer (3) of the transdermal therapeutic system exclusively has non-amine-resistant pressure sensitive silicone adhesives as the pressure sensitive adhesives; i.e., especially the at least one additional initially active ingredient-free pressure sensitive adhesive layer (3) contains no other polymer adhesive, but only non-amine-resistant pressure sensitive silicone adhesives.

In a further preferred embodiment the transdermal therapeutic system according to the invention has a weight percentage of non-amine-resistant pressure sensitive silicone adhesive of more than 50% by weight, preferably more than 60% by weight, more preferably more than 70% by weight, more preferably more than 75% by weight, more preferably more than 80% by weight, more preferably more than 85% by weight, more preferably more than 90% by weight, further preferred more than 93% by weight, further preferred more than 95% by weight, further preferred at least 99% by weight, based on the total weight of the pressure sensitive adhesive of the transdermal therapeutic system (wherein the transdermal therapeutic system can have a single non-amine-resistant pressure sensitive silicone adhesive, or also a mixture of two, three, four etc. non-amine-resistant silicone adhesives). In a further embodiment the transdermal therapeutic system exclusively has non-amine-resistant pressure sensitive silicone adhesives as the pressure sensitive adhesives; i.e., especially the transdermal therapeutic system contains no other polymer adhesive, but only non-amine-resistant pressure sensitive silicone adhesives.

In a further preferred embodiment the transdermal therapeutic system has at least one additional initially active ingredient-free pressure sensitive adhesive layer (3), wherein the at least one additional initially active ingredient-free pressure sensitive adhesive layer (3) or the matrix layer (2) and the at least one additional initially active ingredient-free pressure sensitive adhesive layer (3) have a weight percentage of non-amine-resistant pressure sensitive silicone adhesive of more than 60% by weight, more preferably more than 70% by weight, more preferably more than 75% by weight, more preferably more than 80% by weight, more preferably more than 85% by weight, more preferably more than 90% by weight, further preferred more than 93% by weight, further preferred more than 95% by weight, further preferred at least 99% by weight, based on the total weight of the pressure sensitive adhesive of the at least one additional initially active ingredient-free pressure sensitive adhesive layer (3) or based on the total weight of the pressure sensitive adhesive of the matrix layer (2) and the at least one additional initially active ingredient-free pressure sensitive adhesive layer (3).

The weight per unit area of the matrix layer (2) of the transdermal therapeutic system according to the invention is not particularly limited. In a general embodiment of the invention the matrix layer (2) has a weight per unit area of from 30-70 g/m², preferably 30-60 g/m². The term “weight per unit area” with respect to a layer of the transdermal therapeutic system according to the invention, such as for example the matrix layer (2) or the at least one additional initially active ingredient-free pressure sensitive adhesive layer (3), refers to the weight per unit area of the dry layer, i.e., the weight per unit area of the layer after the solvent has been removed by drying in the preparation of the TTS.

In a preferred embodiment, in which the transdermal therapeutic system contains no other pressure sensitive adhesive layer except the matrix layer (2), the matrix layer (2) has a weight per unit area of from 40-70 g/m², preferably 45-65 g/m², more preferably about 50-60 g/m², more preferably 50-60 g/m². In a further preferred embodiment, in which the transdermal therapeutic system contains no other pressure sensitive adhesive layer except the matrix layer (2), the matrix layer (2) has a weight per unit area of about 50 g/m², or about 60 g/m².

The term “about”, as is used here before numerical values and numerical ranges, means that a value or range designated by it also includes all values lying within ±10% of the given value or range, or within ±5% of the value or range, or in some embodiments within ±1% of the value or range.

In one embodiment of the invention, in which the transdermal therapeutic system in addition to the matrix layer (2) contains at least one additional initially active ingredient-free pressure sensitive adhesive layer (3), the matrix layer (2) has a weight per unit area of from 30-70 g/m², more preferably 40-70 g/m², more preferably 45-65 g/m², more preferably about 50-60 g/m², more preferably 50-60 g/m², for example about 50 g/m² or about 60 g/m²; and the at least one additional initially active ingredient-free pressure sensitive adhesive layer (3) has a weight per unit area of from 15-40 g/m², preferably 20-40 g/m², more preferably 20-35 g/m², more preferably 25-35 g/m², further preferred about 30 g/m².

In the transdermal therapeutic systems according to the invention the matrix layer (2) and, if at least one additional initially active ingredient-free pressure sensitive adhesive layer (3) is present, the at least one additional initially active ingredient-free pressure sensitive adhesive layer (3) contains one or more non-amine-resistant pressure sensitive silicone adhesives in an amount of more than 50% by weight, based on the total weight of the pressure sensitive adhesive of the matrix layer (2), and, if at least one additional initially active ingredient-free pressure sensitive adhesive layer (3) is present, based on the total weight of the pressure sensitive adhesive of the matrix layer (2) or based on the total weight of the at least one additional initially active ingredient-free pressure sensitive adhesive layer (3), respectively.

In a preferred embodiment the matrix layer (1) and/or the at least one additional initially active ingredient-free pressure sensitive adhesive layer (3), if an additional initially active ingredient-free pressure sensitive adhesive layer (3) is present, have exactly one non-amine-resistant pressure sensitive silicone adhesive. That is, in said embodiment exactly one non-amine-resistant pressure sensitive silicone adhesive is contained in the matrix layer (2), in the at least one additional initially active ingredient-free pressure sensitive adhesive layer (3), or in the matrix layer (2) as well as in the at least one additional initially active ingredient-free pressure sensitive adhesive layer (3). In a further preferred embodiment, the pressure sensitive adhesives of the matrix layer (2) and/or the at least one additional initially active ingredient-free pressure sensitive adhesive layer (3), if present, exclusively consist of exactly one non-amine-resistant pressure sensitive silicone adhesive.

In a further preferred embodiment, the one or more non-amine-resistant pressure sensitive silicone adhesives of the matrix layer (2) and/or the at least one additional initially active ingredient-free pressure sensitive adhesive layer (3), if at least one additional initially active ingredient-free pressure sensitive adhesive layer (3) is present, comprise two or more, or three or more, or four or more, etc. different non-amine-resistant pressure sensitive silicone adhesives, wherein preferably at least one (i.e. one or more) of the non-amine-resistant pressure sensitive silicone adhesives has a medium tack and at least one (i.e. one or more) of the non-amine-resistant pressure sensitive silicone adhesives has a high tack. In a further preferred embodiment, the matrix layer (2), and/or the at least one additional initially active ingredient-free pressure sensitive adhesive layer (3), if at least one additional initially active ingredient-free pressure sensitive adhesive layer (3) is present, contains exactly two, or exactly three, or exactly four, etc. different non-amine-resistant pressure sensitive silicone adhesives. In yet a further preferred embodiment, the matrix layer (2) contains exactly one, and the at least one additional initially active ingredient-free pressure sensitive adhesive layer (3), if present, exactly two non-amine-resistant pressure sensitive silicone adhesives.

In yet a further preferred embodiment, the one or more non-amine-resistant pressure sensitive silicone adhesives of the matrix layer (2) and/or the at least one additional initially active ingredient-free pressure sensitive adhesive layer (3), if at least one additional initially active ingredient-free pressure sensitive adhesive layer (3) is present, comprise at least or exactly two, at least or exactly three, at least or exactly four, etc. non-amine-resistant pressure sensitive silicone adhesives of different molecular weights.

In a preferred embodiment of the transdermal therapeutic system of the invention the one or more non-amine-resistant silicone adhesives of the matrix layer (2), and/or the at least one additional initially active ingredient-free pressure sensitive adhesive layer (3), if present, comprise a mixture of at least one non-amine-resistant pressure sensitive silicone adhesive of medium tack, such as for example Dow Corning® BIO-PSA 7-4501, and at least one non-amine-resistant pressure sensitive silicone adhesive of high tack, such as for example Dow Corning® BIO-PSA 7-4601. In a further preferred embodiment of the transdermal therapeutic system of the invention the one or more non-amine-resistant silicone adhesives of the matrix layer (2), and/or the at least one additional initially active ingredient-free pressure sensitive adhesive layer (3), if present, consist of a mixture of a non-amine-resistant pressure sensitive silicone adhesive of medium tack, such as for example Dow Corning® BIO-PSA 7-4501, and a non-amine-resistant pressure sensitive silicone adhesive of high tack, such as for example Dow Corning® BIO-PSA 7-4601.

In a preferred modification of the above embodiments of the invention the one or more non-amine-resistant silicone adhesives of the matrix layer (2), and/or the at least one additional initially active ingredient-free pressure sensitive adhesive layer (3), if present, consist of a mixture of a non-amine-resistant pressure sensitive silicone adhesive of medium tack, such as for example Dow Corning® BIO-PSA 7-4501, and a non-amine-resistant pressure sensitive silicone adhesive of high tack, such as for example Dow Corning® BIO-PSA 7-4601, wherein the matrix layer (2), and/or the at least one additional initially active ingredient-free pressure sensitive adhesive layer (3), if present, have no other pressure sensitive adhesives.

In a preferred embodiment of the present invention, in which the one or more non-amine-resistant silicone adhesives of the matrix layer (2) and/or the at least one additional initially active ingredient-free pressure sensitive adhesive layer (3), if present, contain a mixture of at least one non-amine-resistant pressure sensitive silicone adhesive of medium tack, and at least one non-amine-resistant pressure sensitive silicone adhesive of high tack, or consist of such a mixture, the non-amine-resistant silicone adhesive of medium tack is present in a weight range preferably of from 0.0-55.0% by weight, more preferably 0.0-45.0% by weight, more preferably about 0.0-35.0% by weight, more preferably 0.0-25.0% by weight, and the non-amine-resistant silicone adhesive of high tack in a weight range preferably of from 45.0-100.0% by weight, more preferably 55.0-100.0% by weight, more preferably 65.0-100.0% by weight, more preferably 75.0-100.0% by weight, based on the total weight of silicone adhesive in the matrix layer (2) and/or in the at least one additional initially active ingredient-free pressure sensitive adhesive layer (3), if present. Here, in case of the presence of a matrix layer (2) and at least one additional initially active ingredient-free pressure sensitive adhesive layer (3) in the TTS the weight ratio of non-amine-resistant silicone adhesive of medium tack to non-amine-resistant silicone adhesive of high tack may be the same or different in both layers.

The non-amine-resistant silicone adhesives in the sense of the present invention may be non-silanol-reduced silicone adhesives (i.e., non-amine-resistant silicone adhesives, wherein the silanol groups are not protected by protective groups) or silanol-reduced silicone adhesives (i.e., non-amine-resistant silicone adhesives, wherein the silanol groups are only partially protected by protective groups). Such non-silanol-reduced or only partially silanol-reduced silicone adhesives are known from the prior art. Preferred non-amine-resistant silicone adhesives are non-silanol-reduced non-amine-resistant silicone adhesives, such as for example Dow Corning® BIO-PSA 7-4501, Dow Corning® BIO-PSA 7-4601, Dow Corning® BIO-PSA 7-4502, Dow Corning® BIO-PSA 7-4602, or the silanol-reduced non-amine-resistant silicone adhesives which due to their low silanol reduction still belong to the non-amine-resistant silicone adhesives, such as Dow Corning® BIO-PSA SRS7-4501 and Dow Corning® BIO-PSA SRS7-4601. Especially preferred are the non-amine-resistant silicone adhesives Dow Corning® BIO-PSA 7-4501, Dow Corning® BIO-PSA 7-4601, Dow Corning® BIO-PSA SRS7-4501, and Dow Corning® BIO-PSA SRS7-4601, more preferably are Dow Corning® BIO-PSA SRS7-4501, and Dow Corning® BIO-PSA SRS7-4601.

Additionally, the inventors of the present invention have surprisingly found that by using silanol-reduced non-amine-resistant silicone adhesives instead of non-silanol-reduced non-amine-resistant silicone adhesives adhesion strength of the resulting transdermal therapeutic systems can be increased. The term “silanol-reduced” in the sense of the present invention means that in the so-called non-amine-resistant silicone adhesive a part of the silanol groups is protected by protective groups, i.e., such a silanol-reduced silicone adhesive is still a non-amine-resistant silicone adhesive in the sense of the present invention. Silanol-reduced non-amine-resistant silicone adhesives are known from the prior art and are for example, described in the printed U.S. patent specification U.S. Pat. No. 6,337,086. Dow Corning® BIO-PSA SRS7-4501, Dow Corning® BIO-PSA SRS7-4601, Dow Corning® BIO-PSA SRS7-4502, Dow Corning® BIO-PSA SRS7-4602 are examples of silanol-reduced non-amine-resistant silicone adhesives. Preferred are the silanol-reduced non-amine-resistant silicone adhesives Dow Corning® BIO-PSA SRS7-4501, and Dow Corning® BIO-PSA SRS7-4601. According to a preferred embodiment of the present invention the silanol content of a silanol-reduced non-amine-resistant silicone adhesive in ppm preferably is between about 8000 ppm and 13000 ppm and the silanol content of a non-silanol-reduced non-amine-resistant silicone adhesive is preferably more than 13000 ppm, and, for example as described in U.S. Pat. No. 6,337,086, can be determined for example by means of ²⁹Si-NMR spectroscopy and/or FTIR spectroscopy.

In a preferred embodiment of the present invention the one or more non-amine-resistant silicone adhesives of the matrix layer (2) comprise one or more silanol-reduced non-amine-resistant silicone adhesives, preferably with a weight percentage of silanol-reduced non-amine-resistant silicone adhesive of more than 50% by weight, more preferably more than 60% by weight, more preferably more than 75% by weight, more preferably more than 85% by weight, more preferably more than 90% by weight, more preferably more than 95% by weight, more preferably at least 99% by weight, based on the total weight of the non-amine-resistant silicone adhesive of the matrix layer (2).

In a preferred embodiment of the present invention, in which the transdermal therapeutic system comprises at least one additional initially active ingredient-free pressure sensitive adhesive layer (3), the one or more non-amine-resistant silicone adhesives of the at least one additional initially active ingredient-free pressure sensitive adhesive layer (3) comprise one or more silanol-reduced non-amine-resistant silicone adhesives, preferably with a weight percentage of silanol-reduced non-amine-resistant silicone adhesive of more than 50% by weight, more preferably more than 60% by weight, more preferably more than 75% by weight, more preferably more than 85% by weight, more preferably more than 90% by weight, more preferably more than 95% by weight, more preferably at least 99% by weight, based on the total weight of the non-amine-resistant silicone adhesive of the at least one additional initially active ingredient-free pressure sensitive adhesive layer (3).

For example, the one or more non-amine-resistant silicone adhesives of the matrix layer (2) can also comprise two, three, four, etc. silanol-reduced non-amine-resistant silicone adhesives, preferably with the above-described weight percentages of the total weight of the non-amine-resistant silicone adhesive of the matrix layer (2). Equally, for example the one or more non-amine-resistant silicone adhesives of the at least one additional initially active ingredient-free pressure sensitive adhesive layer (3), too, can comprise two, three, four etc. silanol-reduced non-amine-resistant silicone adhesives, preferably with the above-described weight percentages, but based on the total weight of the non-amine-resistant silicone adhesive of the at least one additional initially active ingredient-free pressure sensitive adhesive layer (3).

As observed by the inventors of the present invention use of a silanol-reduced non-amine-resistant silicone adhesive of high tack such as Dow Corning® BIO-PSA SRS7-4601 even when using a release liner (4) of fluoro-siliconized foil such as for example Scotchpak 9709 (3M Corporation) can result in a significant increase in the separation force during storage. Surprisingly, the present inventors have found that by using a mixture of a silanol-reduced non-amine-resistant silicone adhesive of medium tack, such as for example Dow Corning® BIO-PSA SRS7-4501, and a silanol-reduced non-amine-resistant silicone adhesive of high tack, such as for example Dow Corning® BIO-PSA SRS7-4601, such an increase in the separation during storage can significantly be reduced.

Therefore, in one embodiment according to the invention, in which the one or more non-amine-resistant silicone adhesives of the matrix layer (2) either comprise silanol-reduced non-amine-resistant silicone adhesives or exclusively consist thereof, the matrix layer (2) preferably contains a mixture of one or more silanol-reduced non-amine-resistant pressure sensitive silicone adhesives of medium tack, such as for example Dow Corning® BIO-PSA SRS7-4501, and one or more silanol-reduced non-amine-resistant pressure sensitive silicone adhesives of high tack, such as for example Dow Corning® BIO-PSA SRS7-4601.

In a further embodiment of the present invention, in which the transdermal therapeutic system comprises at least one additional initially active ingredient-free pressure sensitive adhesive layer (3) and in which the one or more non-amine-resistant silicone adhesives of the at least one additional initially active ingredient-free pressure sensitive adhesive layer (3) comprise silanol-reduced non-amine-resistant silicone adhesives or exclusively consist thereof, the at least one additional initially active ingredient-free pressure sensitive adhesive layer (3) preferably contains a mixture of one or more silanol-reduced non-amine-resistant pressure sensitive silicone adhesives of medium tack, such as for example Dow Corning® BIO-PSA SRS7-4501, and one or more silanol-reduced non-amine-resistant pressure sensitive silicone adhesives of high tack, such as for example Dow Corning® BIO-PSA SRS7-4601.

In a preferred embodiment of the present invention, in which the one or more non-amine-resistant silicone adhesives of the matrix layer (2) and/or the at least one additional initially active ingredient-free pressure sensitive adhesive layer (3), if present, contain a mixture of one or more silanol-reduced non-amine-resistant pressure sensitive silicone adhesives of medium tack and one or more silanol-reduced non-amine-resistant pressure sensitive silicone adhesives of high tack or consist of such a mixture, the silanol-reduced non-amine-resistant silicone adhesive of medium tack is present in a weight range preferably of from 10.0-40.0% by weight, more preferably 15.0-35.0% by weight, more preferably about 17.5-30.0% by weight, more preferably 17.5-30.0% by weight, and the silanol-reduced non-amine-resistant silicone adhesive of high tack in a weight range preferably of from 60.0-90.0% by weight, more preferably 65.0-85.0% by weight, more preferably about 70.0-82.5% by weight, more preferably 70.0-82.5% by weight, based on the total weight of silicone adhesive in the matrix layer (2) and/or in the at least one additional initially active ingredient-free pressure sensitive adhesive layer (3), if present. Here, in case of the presence of a matrix layer (2) and at least one additional initially active ingredient-free pressure sensitive adhesive layer (3) in the TTS, the weight ratio of silanol-reduced non-amine-resistant silicone adhesive of medium tack to silanol-reduced non-amine-resistant silicone adhesive of high tack may be the same or different in both layers.

In a preferred modification of the above-mentioned embodiments of the transdermal therapeutic system with silanol-reduced non-amine-resistant pressure sensitive silicone adhesive the one or more non-amine-resistant pressure sensitive silicone adhesives of the matrix layer (2) and/or the at least one additional initially active ingredient-free pressure sensitive adhesive layer (3), if present, exclusively consist of a silanol-reduced non-amine-resistant pressure sensitive silicone adhesive. In a further preferred modification of the above-mentioned embodiments the matrix layer (2) and/or the at least one additional initially active ingredient-free pressure sensitive adhesive layer (3), if present, exclusively have silanol-reduced non-amine-resistant pressure sensitive silicone adhesives as pressure sensitive adhesives.

In yet a further preferred modification of the above-mentioned embodiments of the transdermal therapeutic system with silanol-reduced non-amine-resistant pressure sensitive silicone adhesives the pressure sensitive adhesives of the matrix layer (2) and/or the at least one additional initially active ingredient-free pressure sensitive adhesive layer (3), if present, exclusively consist of a mixture of a silanol-reduced non-amine-resistant pressure sensitive silicone adhesive of medium tack, such as for example Dow Corning® BIO-PSA SRS7-4501, and a silanol-reduced non-amine-resistant pressure sensitive silicone adhesive of high tack, such as for example Dow Corning® BIO-PSA SRS7-4601.

In a preferred embodiment of a bilayer formulation according to the invention the one or more non-amine-resistant silicone adhesives of the matrix layer (2) exclusively consist of a silanol-reduced non-amine-resistant pressure sensitive silicone adhesive of medium tack, such as for example Dow Corning® BIO-PSA SRS7-4501, and the one or more non-amine-resistant silicone adhesives of the at least one additional initially active ingredient-free pressure sensitive adhesive layer (3) exclusively consist of a mixture of a silanol-reduced non-amine-resistant pressure sensitive silicone adhesive of medium tack, such as for example Dow Corning® BIO-PSA SRS7-4501, and a silanol-reduced non-amine-resistant pressure sensitive silicone adhesive of high tack, such as for example Dow Corning® BIO-PSA SRS7-4601. In a further preferred embodiment of a bilayer formulation according to the invention the pressure sensitive adhesives of the matrix layer (2) exclusively consist of a silanol-reduced non-amine-resistant pressure sensitive silicone adhesive of medium tack, and the pressure sensitive adhesives of the at least one additional initially active ingredient-free pressure sensitive adhesive layer (3) exclusively consist of a mixture of a silanol-reduced non-amine-resistant pressure sensitive silicone adhesive of medium tack and a silanol-reduced non-amine-resistant pressure sensitive silicone adhesive of high tack.

In a preferred modification of the above embodiments with one or more silanol-reduced non-amine-resistant silicone adhesives one or more non-silanol-reduced non-amine-resistant silicone adhesives are used instead of the one or more silanol-reduced non-amine-resistant silicone adhesives. That is, for example in a preferred embodiment of the present invention the one or more non-amine-resistant silicone adhesives of the matrix layer (2), and/or the at least one additional initially active ingredient-free pressure sensitive adhesive layer (3), if present, comprise one or more non-silanol-reduced non-amine-resistant silicone adhesives, preferably with a weight percentage of non-silanol-reduced non-amine-resistant silicone adhesive of more than 50% by weight, more preferably more than 60% by weight, more preferably more than 75% by weight, more preferably more than 85% by weight, more preferably more than 90% by weight, more preferably more than 95% by weight, more preferably at least 99% by weight, based on the total weight of the non-amine-resistant silicone adhesive of the matrix layer (2), and/or the at least one additional initially active ingredient-free pressure sensitive adhesive layer (3), if present. Or for example, in a preferred modification of the above-mentioned embodiments the one or more non-amine-resistant pressure sensitive silicone adhesives of the matrix layer (2) and/or the at least one additional initially active ingredient-free pressure sensitive adhesive layer (3), if present, exclusively consist of a non-silanol-reduced non-amine-resistant pressure sensitive silicone adhesive.

For example, the one or more non-amine-resistant silicone adhesives of the matrix layer (2), and/or the one or more non-amine-resistant silicone adhesives of the at least one additional initially active ingredient-free pressure sensitive adhesive layer (3), if at least one such initially active ingredient-free pressure sensitive adhesive layer (3) is present, can also comprise two, three, four, etc. non-silanol-reduced non-amine-resistant silicone adhesives, preferably with the above-described weight percentages of the total weight of the non-amine-resistant silicone adhesives of the matrix layer (2), or the at least one initially active ingredient-free pressure sensitive adhesive layer (3). Non-silanol-reduced non-amine-resistant silicone adhesives are known to the skilled person. Preferred non-silanol-reduced non-amine-resistant silicone adhesives are Dow Corning® BIO-PSA 7-4501, Dow Corning® BIO-PSA 7-4601, Dow Corning® BIO-PSA 7-4502, and Dow Corning® BIO-PSA 7-4602.

All the above embodiments may preferably be designed such that the matrix layer (2) of the transdermal therapeutic system, and/or the at least one additional initially active ingredient-free pressure sensitive adhesive layer (3), if present, and/or the entire transdermal therapeutic system exclusively contains silicone adhesives as pressure sensitive adhesives. In addition, all the above embodiments may preferably be designed such that the matrix layer (2) of the transdermal therapeutic system, and/or the at least one additional initially active ingredient-free pressure sensitive adhesive layer (3), if present, and/or the entire transdermal therapeutic system exclusively contains non-amine-resistant pressure sensitive silicone adhesives as pressure sensitive silicone adhesives; i.e. especially the matrix layer (2) and/or the at least one additional initially active ingredient-free pressure sensitive adhesive layer (3), if present, and/or the entire transdermal therapeutic system contains no amine-resistant pressure sensitive silicone adhesive, but exclusively non-amine-resistant pressure sensitive silicone adhesives.

The application period of the transdermal therapeutic system according to the invention preferably is one day, i.e., the transdermal therapeutic system according to the invention is removed from the skin after one day. Since the transdermal therapeutic systems according to the invention in general are used for long-term treatment (several months or even several years) a new transdermal therapeutic system according to the invention is adhered to the skin after having removed a transdermal therapeutic system after one day.

However, the transdermal therapeutic systems of the invention may also be used for more than one day, e.g., 2 days or 3 days. In these cases, a transdermal therapeutic system according to the invention is replaced by a new transdermal therapeutic system after 2 or 3 days, respectively.

There is a backing layer (1) on the side of the adhesive matrix facing away from the human skin in use which in a more preferred embodiment is occlusive for the active ingredient, i.e., not permeable. It is also particularly preferred that the backing layer is largely impermeable to light. In one embodiment such backing layers can consist of polyesters, polyolefins, in particular polyethylene, or polyurethanes. Backing layers, containing several different polymers arranged above each other can also advantageously be used. Preferably, the backing layer has a high imperviousness to water vapor.

A preferred material for the backing layer is polyester, for example in the form of a composite foil with polyester inside, a central aluminum barrier and pigmented polyethylene on the outside. Particularly preferred backing layers are for example the polyester-based foils sold by 3M under the designation Scotchpak 1109 or Scotchpak 9738 or the polyester-based foils sold by Mitsubishi Polyester Film under the designation Hostaphan® MN19, Hostaphan® MN 19 Med, and Hostaphan® MN 15 Med; particularly preferred for a monolayer formulation is for example Scotchpak 9738 and for a bilayer formulation for example Hostaphan® MN 19 Med.

Other suitable materials comprise cellophane, cellulose acetate, ethyl cellulose, vinyl acetate vinyl chloride copolymers provided with plasticizers, ethylene vinyl acetate copolymers, polyethylene terephthalate, nylon, polyethylene, polypropylene, polyvinylidene chloride, ethylene methacrylate copolymer, paper which can optionally be coated, textile fabric, such as polyethylene terephthalate foils, aluminum foils, and polymer metal composite materials.

The thickness of the backing layer (1) of the transdermal therapeutic systems according to the invention is not particularly limited. In a preferred embodiment the backing layer (1) comprises a polyester foil, preferably with a thickness below 35 μm, more preferably 5-30 μm, more preferably 10-25 μm, particularly preferred 15-23 μm. In a further embodiment the backing layer (1) consists of a polyester foil, preferably with a thickness below 70 μm, more preferably 15-65 μm, more preferably 25-60 μm, particularly preferred 30-60 μm, alternatively particularly preferred 49-60 μm, further alternatively particularly preferred 31-37 μm.

On the backing layer (1) of the plaster there can also be a cover layer which is particularly to prevent the plaster from adhering to the package, in case that small amounts of the matrix material escape. The cover layer preferably lies lose on the backing layer and is held by electrostatic forces. Such cover layers are known in the prior art, e.g., from EP 1 097 090, to which reference is fully made on all points. The cover layer at least on the side lying on the backing layer is non-stick, e.g., fluorinated or fluoro-siliconized.

Further, the transdermal therapeutic system according to the invention comprises a release liner (4) to be removed before use. The release liner (4) follows the matrix layer (2), or if at least one additional initially active ingredient-free pressure sensitive adhesive layer (3) is present the at least one additional initially active ingredient-free pressure sensitive adhesive layer (see for example, FIG. 1 (A) and (B), respectively). Here, the release liner (4) to be removed before use preferably is the outer layer of the TTS, so that one side of the release liner (4) to be removed before use forms an outer surface. If there is no additional initially active ingredient-free pressure sensitive adhesive layer (3) present the release liner (4) to be removed before use preferably is in direct contact with the matrix layer (2), so that the opposite of the release liner (4) to be removed before use represents the outer surface of the TTS. If an additional initially active ingredient-free pressure sensitive adhesive layer (3) is present, the additional initially active ingredient-free pressure sensitive adhesive layer (3) is between the matrix layer (2) and the release liner (4) to be removed before use and preferably is in direct contact with the matrix layer (2) and/or the release liner (4) to be removed before use. If more than one additional initially active ingredient-free pressure sensitive adhesive layer (3) is present, the several additional initially active ingredient-free pressure sensitive adhesive layers (3) are between the matrix layer (2) and the release liner (4) to be removed before use, so that the release liner (4) to be removed before use preferably is in direct contact with that additional initially active ingredient-free pressure sensitive adhesive layer (3) which is furthest away from the matrix layer (2).

The release liner (4) to be removed before use preferably is made of a polymeric material which can optionally also be metallized. Examples of polymeric materials that are preferably used are polyesters, polyurethanes, polyvinyl acetate, polyvinylidene chloride, polypropylene, polycarbonate, polystyrene, polyethylene, polyethylene terephthalate, polybutylene terephthalate as well as paper which is optionally surface coated with corresponding polymers. Preferably, it is a release liner (4) which is fluoro-siliconized on one side or on both sides. Particularly preferred are commercially fluoro-siliconized polyester foils, such as the fluoro-siliconized trading product Scotchpak 9709 (3M). In a preferred embodiment the transdermal therapeutic system further comprises a release liner (4) to be removed before use consisting of a fluoro-siliconized foil, preferably a fluoro-siliconized polyester foil.

In a preferred embodiment the transdermal therapeutic system according to the invention consists of the backing layer (1); the matrix layer (2) which is on the backing layer; and the release liner (4) to be removed before use which is on the matrix layer.

In a further preferred embodiment, the transdermal therapeutic system consists of the backing layer (1); the matrix layer (2) which is on the backing layer; the at least one additional initially active ingredient-free pressure sensitive adhesive layer (3) that is between the matrix layer and the release liner (4) to be removed before use; and the release liner (4) to be removed before use that is on the at least one additional initially active ingredient-free pressure sensitive adhesive layer.

According to the invention the active ingredient is in the matrix layer (2). The active ingredient is rotigotine or a pharmaceutically acceptable salt of rotigotine, preferably rotigotine. In view of the polymorphic forms of rotigotine which can be used the invention is not limited, however, for reasons of stability rotigotine of the polymorphic form II, as described in WO 2009/068520, is preferred. In view of the preparation and characterization of rotigotine of the polymorphic form II fully reference is made to WO 2009/068520.

Preferably, the active ingredient is present in the matrix layer (the adhesive matrix) completely dissolved, i.e., the matrix layer preferably contains no solid active ingredient particles. The rotigotine content in the matrix layer (2) is preferably in the range of from 5% by weight to 25% by weight, more preferably in the range of from 6% by weight to 20% by weight, more preferably in range of from 6% by weight to 15% by weight, more preferably in the range of from 6.5% by weight to 11.5% by weight, for example 6.875-9% by weight, especially about 7.5-9% by weight, of rotigotine, based on the total weight of the matrix layer (2).

According to the invention, the active ingredient is present in the matrix layer (the adhesive matrix) preferably in the dispersed phase of a solid dispersion substantially in a non-crystalline form, wherein the one or more non-amine-resistant silicone adhesives as well as possible other polymer adhesives preferably form the dispersant. In a preferred embodiment the dispersed phase in addition to the non-crystalline rotigotine comprises a polyvinyl pyrrolidone. In this context, substantially means to more than 50%, especially to more than 90%, particularly preferred to more than 99% or completely.

Rotigotine is very poorly soluble in silicone adhesives, but good soluble e.g., in a crystallization inhibitor such as polyvinylpyrrolidone. In a preferred embodiment of the transdermal therapeutic system according to the invention the matrix layer (2) thus in addition to the non-amine-resistant silicone adhesives has also polyvinylpyrrolidone dispersed therein. Rotigotine is preferably completely dissolved in the matrix layer, i.e. the content of rotigotine in the silicone adhesive is so small that preferably no rotigotine precipitates/crystalizes and the major part of the rotigotine preferably is present dissolved (or at least in a non-crystalline form) in the dispersed polyvinylpyrrolidone.

Polyvinylpyrrolidone (PVP) is a polymer from the monomer N-vinylpyrrolidone. It is known to be able to increase cohesion of silicone adhesives. Polyvinylpyrrolidone can also serve as a crystallization inhibitor for the active ingredient rotigotine. The molecular weight of the polyvinylpyrrolidone may be in the range of from 2,000 to 2,500,000 Dalton (g/mol) (given as the average weight), preferably in a range of from 700,000 to 1,500,000 Dalton, more preferably in a range of from 900,000 to 1,500,000 Dalton. Various PVP qualities are available, for example from BASF AG, Ludwigshafen, Germany, e.g., under the name Kollidon. For example, the following Kollidon grades are water-soluble forms of PVP: K-12 PF (molecular weight=2,000-3,000 Dalton); K-17 PF (molecular weight=7,000-11,000 Dalton); K-25 (molecular weight=28,000-34,000 Dalton); K-30 (molecular weight=44,000-54,000 Dalton); and K-90 (molecular weight=900,000-1,500,000 Dalton). In a preferred embodiment the molecular weight of the polyvinylpyrrolidone is in the range of from 28,000 to 1,500,000 Dalton (g/mol).

The present inventors have surprisingly found that in a transdermal therapeutic system with the active ingredient rotigotine and one or more non-amine-resistant silicone adhesives in the matrix layer in an amount of more than 50% by weight, based on the total weight of the pressure sensitive adhesive of the matrix layer (2) when using rotigotine and a polyvinylpyrrolidone in the matrix layer in the dispersed phase of a solid dispersion in a weight ratio of from 9:6.4, especially 9:7 or less no crystallization takes place even after longer storage times at 25° C. or higher. On the other hand, with a larger weight ratio of rotigotine and polyvinylpyrrolidone, for example of from 9:5 or larger, there is the risk that crystallization can occur after longer storage times at temperatures of 25° C. or higher. Such high weight ratios of rotigotine to polyvinylpyrrolidone therefore according to the invention certainly are possible, but not preferred.

In a preferred embodiment of the transdermal therapeutic systems according to the invention therefore rotigotine is present in the matrix layer substantially in a non-crystalline form in the dispersed phase of a solid dispersion comprising a polyvinylpyrrolidone (PVP), wherein the weight ratio of rotigotine to polyvinylpyrrolidone is at most 9:6.4, more preferably at most 9:6.5, especially at most 9:7. The weight ratio of rotigotine to polyvinylpyrrolidone is preferably at least 9:11, more preferably at least 9:10, especially at least 9:9. Preferably, the ratio is in the range of from 9:7 to 9:10. With such a weight ratio the matrix layer can contain for example 5.14-12.86% by weight of rotigotine and 4-10% by weight of polyvinylpyrrolidone, or 6.88-9% by weight of rotigotine and 5.35-7% by weight of polyvinylpyrrolidone, based on the total weight of the matrix layer. In further preferred embodiments of the present invention at least 70% by weight, at least 80% by weight, at least 85% by weight, at least 90% by weight, at least 95% by weight, at least 97.5% by weight, of rotigotine are present in the matrix layer in a non-crystalline form in the dispersed phase of a solid dispersion comprising a polyvinylpyrrolidone (PVP).

Appropriate polyvinylpyrrolidones for use in combination with rotigotine in the matrix layer of a transdermal therapeutic system are known from the prior art. Such polyvinylpyrrolidones are described for example in WO 2011/076879. Preferred polyvinylpyrrolidones with respect to the present invention are PVP K90 (BASF SE). Particularly preferred is the polyvinylpyrrolidone type K-90 (PVP K90).

The matrix layer (2) and/or the at least one additional initially active ingredient-free pressure sensitive adhesive layer (3) can contain further polymer adhesives (i.e., pressure sensitive adhesives) that are not non-amine-resistant silicone adhesives. The weight percentage of further polymer adhesives which are not non-amine-resistant silicone adhesives in the matrix layer (2) is less than 50% by weight, preferably less than 40% by weight, more preferably less than 30% by weight, more preferably less than 20% by weight, more preferably less than 10% by weight, more preferably less than 5% by weight, based on the total weight of the pressure sensitive adhesive of the matrix layer (2). If there is at least one additional initially active ingredient-free pressure sensitive adhesive layer (3) present in the TTS, the weight percentage of further polymer adhesives which are not non-amine-resistant silicone adhesives in the at least one additional initially active ingredient-free pressure sensitive adhesive layer (3) is less than 50% by weight, preferably less than 40% by weight, more preferably less than 30% by weight, more preferably less than 20% by weight, more preferably less than 10% by weight, more preferably less than 5% by weight, based on the total weight of the at least one additional initially active ingredient-free pressure sensitive adhesive layer (3). Such further polymer adhesives are for example polyacrylates, polymethacrylates, SBS block copolymers, and polyisobutylenes.

Polyacrylates and polymethacrylates are known in the prior art (cf., for example U.S. 2002/0077437) and are used in many ways for transdermal therapeutic systems. Polyacrylates or polymethacrylates, respectively, generally are prepared by radical polymerization of acrylic or methacrylic acid derivatives, especially acrylic or methacrylic acid esters, wherein also other suitable compounds such as e.g., vinyl acetate can be copolymerized as additional monomers. The polyacrylates or polymethacrylates can be cross-linked, for example by polyvalent metal ions to modify the properties of the polyacrylates or polymethacrylates. Both cross-linked and uncross-linked polyacrylates or polymethacrylates are commercially available, one of the main supplier is Henkel (or National Starch) which sells the polyacrylates and polymethacrylates under the designation “DURO-TAK”.

Examples are polyacrylates or polymethacrylate copolymers or terpolymers of monomers which are selected for example from acrylic acid, methacrylic acid, methoxy ethyl acrylate, ethyl acrylate, ethyl methacrylate, butyl acrylate, butyl methacrylate, hexyl acrylate, hexyl methacrylate, methyl acrylate, methyl methacrylate, 2-ethyl butyl acrylate 2-ethyl butyl methacrylate, isooctyl acrylate, isooctyl methacrylate, 2-ethyl hexyl acrylate, 2-ethyl hexyl methacrylate, decyl acrylate, decyl methacrylate, dodecyl acrylate, dodecyl methacrylate, tridecyl acrylate, tridecyl methacrylate, hydroxy ethyl acrylate, hydroxy propyl acrylate, dimethyl amino ethyl acrylate, dimethyl amino ethyl methacrylate, tertbutyl amino ethyl acrylate, tertbutyl amino ethyl methacrylate, methoxy ethyl acrylate, methoxy ethyl methacrylate, etc. Optionally, as the comonomers also acrylamide, dimethyl amide, acrylonitrile, and vinyl acetate can be used. Further examples of suitable acryl adhesives are mentioned for example in Satas, “Acrylic Adhesives, Handbook of Pressure Sensitive Adhesives Technology, 2^{nd} edition, pages 396-456 (D. Satas, editor) van Nostrand Reinhold, New York (1989). Wherever polyacrylates are mentioned herein, the corresponding polymethacrylates are also meant.

Polyisobutylenes are known in the prior art and commercially available. For example, the products Oppanol sold by BASF, Ludwigshafen, Germany. Suitable polyisobutylenes are for example Oppanol B50, N50, B80, N80, B100, N100, B150, N150, B200, and N200 or also Oppanol B10SFN or Oppanol B15SFN B10, B15. For example, also mixtures of a polyisobutylene selected for example from Oppanol B80, Oppanol B100, Oppanol B150, and Oppanol B200, preferably Oppanol B80 or Oppanol B100, with a second polyisobutylene selected from the products Oppanol B10SFN and Oppanol B15SFN, can be used.

Insofar as the molecular weight of polymers is referred to in the context of this invention, it is always the weight average molecular weight M_{w}, unless explicitly stated otherwise or obvious due to the context. The weight average molecular weight M_{w} for example can be determined by GPC, as is known to the skilled person.

In the transdermal therapeutic systems according to the invention the matrix layer containing the active ingredient in addition to the mentioned pressure sensitive adhesives, rotigotine and optionally polyvinylpyrrolidone can contain other components if necessary.

For example, a penetration enhancer can be added to the matrix layer to ensure sufficient permeation of the active ingredient through the skin. Suitable penetration enhancers are known. For example, these are fatty alcohols, fatty acids, fatty acid esters, fatty acid amides, glycerin, and glycerin derivatives, n-methyl pyrrolidone, terpene and terpene derivatives, such as D-limonene, α-pinene, a-terpineol, carvone, carveol, limonene oxide, pinene oxide, 1,8-eucalyptol. However, preferably the transdermal therapeutic system according to the invention contains no such penetration enhancers.

Moreover, optionally one or more plasticizers can be added to the matrix layer (2). Suitable plasticizers are also known in the prior art and here, for example plasticizers on the basis of mineral oil or polybutene can be mentioned. In one embodiment the matrix layer (2) and/or the at least one additional initially active ingredient-free pressure sensitive adhesive layer (3), if present, contain one or more additives, preferably a plasticizer.

In a preferred embodiment of the invention the matrix layer (2) also contains one or more additives to improve the chemical stability of rotigotine, e.g., antioxidants, such as tocopherol and its derivatives, especially esters, butyl hydroxy toluoyl (BHT), butyl hydroxy anisole (BHA), ascorbic acid and its derivatives, especially esters and/or sodium metabisulfite. In one embodiment the matrix layer (2) contains tocopherol, ascorbyl palmitate and sodium metabisulfite, for example about 0.05-0.125% by weight of tocopherol, 0.0-0.1% by weight of ascorbyl palmitate, and 0.0-0.0021% by weight of sodium metabisulfite, based on the total weight of the matrix layer (2). Preferably, no antioxidants or sodium metabisulfite are added to the at least one additional initially active ingredient-free pressure sensitive adhesive layer (3).

The transdermal therapeutic system according to the invention can be used to treat all diseases in which administration of the active ingredient rotigotine is indicated. However, particularly preferred the transdermal therapeutic system according to the invention is used for the treatment of the Parkinson disease.

The transdermal therapeutic system according to the invention can be prepared in a manner known per se. For example, for a monolayer formulation all components of the matrix layer of the transdermal therapeutic system are added to a suitable solvent and stirred to the desired homogeneity. Subsequently, the homogenized coating mass is applied to the backing layer (1) or preferably to a release liner (4) and the solvent is removed by drying. Finally, the remaining layer, i.e., the release liner (4) or preferably the backing layer (1) is laminated onto the matrix layer (2) and transdermal therapeutic systems of a suitable size are blanked.

Thus, the present invention according to a preferred embodiment is further directed to a method for the preparation of a transdermal therapeutic system as a monolayer formulation according to one of the above-described embodiments of the transdermal therapeutic system according to the invention comprising a) preparing a homogenized coating mass by adding all components of the matrix layer (2) of the transdermal therapeutic system together in a suitable solvent and mixing to the desired homogeneity; b) applying the homogenized coating mass onto a backing layer (1) or preferably onto a release liner (4) and removing the solvent by drying; and c) laminating the remaining layer, i.e. a release liner (4) or preferably a backing layer (1), onto the matrix layer (2), and blanking transdermal therapeutic systems of the suitable size.

For a bilayer formulation or multilayer formulation, respectively for example all the components of the active ingredient-containing matrix layer (2) of the transdermal therapeutic system are added together in a suitable solvent and stirred to the desired homogeneity. Subsequently, said first stirred or homogenized coating mass is applied to the backing layer (1), or preferably to a provisional release liner (4), and the solvent is removed by drying. Finally, the remaining layer, i.e., the provisional release liner (4) or preferably the backing layer (1) is laminated onto the matrix layer (2) (first precursor of the TTS). Then, the components of the at least one additional initially active ingredient-free pressure sensitive adhesive layer (3) of the transdermal therapeutic system are added together in a suitable solvent and stirred to the desired homogeneity. Subsequently, said further coating mass is applied to a release liner (4) and the solvent is removed by drying (second precursor of the TTS). Finally, the provisional release liner (4) is peeled off from the first precursor of the TTS and laminated with the at least one additional initially active ingredient-free pressure sensitive adhesive layer (3) along with the release liner (4) (second precursor of the TTS) to an overall laminate comprising or consisting of in this order the backing layer (1), the active ingredient-containing matrix layer (2), at least one additional initially active ingredient-free pressure sensitive adhesive layer (3) and release liner (4) and transdermal therapeutic systems of the suitable size are blanked from the overall laminate.

Thus, the present invention according to a second preferred embodiment is further directed to a method for the preparation of a transdermal therapeutic system comprising a) preparing a first precursor of the transdermal therapeutic system comprising a1) preparing a first homogenized coating mass by adding together all the components of the matrix layer (2) in a suitable solvent and mixing to the desired homogeneity; a2) applying the first homogenized coating mass onto a backing layer (1), or preferably onto a provisional release liner (4), and removing the solvent by drying; and a3) laminating the remaining layer, i.e. a provisional release liner (4) or preferably a backing layer (1), onto the matrix layer (2); b) preparing a second precursor of the transdermal therapeutic system comprising b1) preparing a further homogenized coating mass by adding together all the components of the at least one additional initially active ingredient-free pressure sensitive adhesive layer (3) in a suitable solvent and mixing to the desired homogeneity; b2) applying the further homogenized coating mass onto a release liner (4) and removing the solvent by drying; and c) removing the provisional release liner (4) from the first precursor of the transdermal therapeutic system of a), laminating the first precursor and the second precursor of the transdermal therapeutic system to an overall laminate comprising or consisting of in this order a backing layer (1), an active ingredient-containing matrix layer (2), at least other additional initially active ingredient-free pressure sensitive adhesive layer (3) and a release liner (4), and blanking transdermal therapeutic systems of the suitable size.

In the above-described methods for the preparation of a transdermal therapeutic system further layers can be inserted by further intermediate process steps in a manner known per se. For example, a membrane for controlling the active ingredient release can be inserted between the matrix layer and the at least one additional initially active ingredient-free pressure sensitive adhesive layer (3). Or, for example a TTS comprising at least two additional initially active ingredient-free pressure sensitive adhesive layers (3) can be prepared by removing the release liner (4) for example from a bilayer formulation of the TTS prepared according to the above second preferred embodiment of the method for the preparation of the TTS according to the invention, and laminating said bilayer formulation with a further second precursor of the TTS, for example as prepared in step b) (of the above second preferred embodiment of the method for the preparation of the TTS according to the invention), to an overall laminate comprising in this order of layers a backing layer (1), an active ingredient-containing matrix layer (2), a first and second additional initially active ingredient-free pressure sensitive adhesive layer (3) and a release liner (4), and blanking transdermal therapeutic systems of the suitable size. In this way, further layers can be inserted into the TTS.

### EXAMPLES

The following examples explain the invention. Percentages always mean percentage by weight.

### Example 1 a: Monolayer (Preparation of the Test Formulations Using the Example of 616ROTTS)

| | Composition of the | Composition of the |
|---|---|---|
| Raw Material | dried matrix [%] | wet matrix [g/m²] |
| Rotigotine | 7.50 | 4.50 |
| PVP K90 | 5.83 | 3.50 |
| Ethanol | — | 19.82 |
| Sodium metabisulfite | 0.0021 | 0.0013 |
| Water, dist. | — | 0.1247 |
| Ascorbyl palmitate | 0.02 | 0.01 |
| Tocopherol | 0.05 | 0.03 |
| Paraffin, viscous | 2.10 | 1.26 |
| BIO-PSA ® SRS7-4501 | 21.12 | 19.22 |
| (e.g., 66% solid content) | | |
| BIO-PSA ® SRS7-4601 | 63.37 | 60.51 |
| (e.g., 63% solid content) | | |
| n-Heptane | — | 2.14 |
| Sum | 100.0 | 111.1 |

An aqueous 1% (w/w %) sodium metabisulfite solution was prepared. An ethanolic 25% (w/w %) PVP K90 solution was prepared. The corresponding amount of sodium metabisulfite solution was added to the corresponding amount of PVP solution in a suitable glass vessel and stirred for ca. 15 min. Ascorbyl palmitate and tocopherol were added and stirred. Then, the active ingredient rotigotine was slowly added under stirring and heating in a water bath and stirred at approximately 60° C. until complete dissolution. After the mass had cooled down again the silicone adhesives were sequentially added and briefly stirred. Then, the balancing solvent heptane was added and stirred. At the end, paraffin is added. The coating mass was stirred until all was visually homogeneously distributed. Subsequently, the coating mass was processed for e.g., approximately 3 minutes at ca. 10000 rpm with a suitable dispersing instrument (Ultra-Turrax). The thus homogenized coating mass was spread out on a fluorine-coated foil, e.g., Scotchpak™ 9709/1022/9744, as a thin film and subsequently heated for e.g., 10 min. at 85° C., so that the solvent was almost completely removed. The dried matrix was about 60 g/m² and was laminated with a release liner, e.g., of polyethylene terephthalate (PET) in a thickness of 19 μm or of polyethylene aluminum polyester.

### Example 1 b: Monolayer (Preparation of the Test Formulations Using the Example of IMPD 631ROTTDS)

| | Composition of the | Composition of the |
|---|---|---|
| Raw Material | dried matrix [%] | wet matrix [g/m²] |
| Rotigotine | 7.50 | 3.75 |
| PVP K90 | 5.83 | 2.92 |
| Ethanol | — | 16.53 |
| Sodium metabisulfite | 0.0001 | 0.0005 |
| Water, dist. | — | 0.0495 |
| Ascorbyl palmitate | 0.05 | 0.03 |
| Tocopherol | 0.05 | 0.03 |
| Paraffin, viscous | 2.10 | 1.05 |
| BIO-PSA ® SRS7-4501 | 21.12 | 15.51 |
| (e.g., 68% solid content) | | |
| BIO-PSA ® SRS7-4601 | 63.35 | 48.99 |
| (e.g., 65% solid content) | | |
| n-Heptane | — | 0.45 |
| Sum | 100.0 | 89.3 |

An aqueous 1% (w/w %) sodium metabisulfite solution was prepared. An ethanolic 25% (w/w %) PVP K90 solution was prepared. The corresponding amount of sodium metabisulfite solution was added to the corresponding amount of PVP solution in a suitable glass vessel and stirred for ca. 30 min. Ascorbyl palmitate and tocopherol were added and stirred. Then, the active ingredient rotigotine was slowly added under stirring and heating in a water bath and stirred at approximately 60° C. until complete dissolution. After the mass had cooled down again the silicone adhesives were sequentially added and briefly stirred. Then, the balancing solvent heptane was added and stirred. At the end, paraffin was added. The coating mass was stirred until all was visually homogeneously distributed. Subsequently, the coating mass was processed for e.g., approximately 3 minutes at ca. 10000 rpm with a suitable dispersing instrument (Ultra-Turrax). The thus homogenized coating mass was spread out on a fluorine-coated foil, e.g., Scotchpak™ 9709, as a thin film and subsequently heated in a drying tunnel of ca. 52 cm in length with 4 separate sections each at ca. 45, 60, 80, 99° C. with a rate of ca. 0.16 m/min, so that the solvents were almost completely removed. The dried matrix was about 50 g/m² and was laminated with a release liner, e.g., of polyethylene terephthalate (PET) in a thickness of 19 μm.

### Example 2 a: Bilayer (Preparation of the Test Formulations Using the Example of 618_617ROTTDS)

| | | |
|---|---|---|
| | Composition of the | Composition of the |
| Raw Material | dried matrix [%] | wet matrix [g/m²] |

Matrix Layer

| | | |
|---|---|---|
| Rotigotine | 9.00 | 4.50 |
| PVP K90 | 7.00 | 3.50 |
| Ethanol | — | 19.83 |
| Sodium metabisulfite | 0.0021 | 0.0011 |
| Water, dist. | — | 0.1040 |
| Ascorbyl palmitate | 0.02 | 0.01 |
| Tocopherol | 0.05 | 0.03 |
| BIO-PSA ® SRS7-4501 | 83.93 | 63.63 |
| (e.g., 66% solid content) | | |
| n-Heptane | — | 0.99 |
| Sum | 100.0 | 92.6 |

Initially active ingredient-free adhesive layer

| | | |
|---|---|---|
| Paraffin, viscous | 2.40 | 0.66 |
| BIO-PSA ® SRS7-4501 | 24.40 | 10.17 |
| (e.g., 66% solid content) | | |
| BIO-PSA ® SRS7-4601 | 73.20 | 32.03 |
| (e.g., 63% solid content) | | |
| n-Heptane | — | 2.97 |
| Sum | 100.0 | 45.8 |

First, the matrix layer (2) was prepared. For that, an aqueous 1% (w/w %) sodium metabisulfite solution as well as an ethanolic 25% (w/w %) PVP K90 solution were prepared. The corresponding amount of sodium metabisulfite solution was added to the corresponding amount of PVP solution in a suitable glass vessel and stirred for at least 15 min. Ascorbyl palmitate and tocopherol were added and stirred. Then, the active ingredient rotigotine was slowly added under stirring and heating in a water bath and stirred at approximately 60° C. until complete dissolution. After the mass had cooled down again the silicone adhesives were sequentially added and briefly stirred. Then, the balancing solvent heptane was added and stirred. The coating mass was stirred until all was visually homogeneously distributed. Subsequently, the coating mass was spread out on a fluorine-coated foil, e.g., Scotchpak™ 9709/1022/9744, as a thin film and subsequently heated for e.g., 10 min. at 85° C., so that the solvents were almost completely removed. The dried matrix was about 50 g/m² and was laminated with a release liner, e.g., of polyethylene terephthalate (PET) in a thickness of 19 μm.

For the preparation of the initially active ingredient-free adhesive layer (3) the silicone adhesives were added into a suitable glass vessel and briefly stirred. Then, paraffin and the balancing solvent heptane were sequentially added and stirred. The coating mass was stirred until all was visually homogeneously distributed. Thereafter, the coating mass was spread out on a fluorine-coated foil, e.g., Scotchpak™ 9709/1022/9744, as a thin film and subsequently heated for e.g., 10 min. at 85° C., so that the solvents were almost completely removed. The dried initially active ingredient-free adhesive layer (3) had a matrix weight in the range of from 25-30 g/m³ and was laminated with the matrix layer onto the open matrix of the initially active ingredient-free adhesive layer (3) while peeling off its release liner.

### Example 2 b: Bilayer (Preparation of the Test Formulations Using the Example of 629_628ROTTDS)

| | | |
|---|---|---|
| | Composition of the | Composition of the |
| Raw Material | dried matrix [%] | wet matrix [g/m²] |

Matrix Layer

| | | |
|---|---|---|
| Rotigotine | 9.00 | 4.50 |
| PVP K90 | 7.00 | 3.50 |
| Ethanol | — | 19.83 |
| Sodium metabisulfite | 0.0021 | 0.0011 |
| Water, dist. | — | 0.1040 |
| Ascorbyl palmitate | 0.10 | 0.05 |
| Tocopherol | 0.05 | 0.03 |
| BIO-PSA ® SRS7-4501 | 83.85 | 62.57 |
| (e.g., 67% solid content) | | |
| n-Heptane | — | 0.32 |
| Sum | 100.0 | 90.9 |

Initially active ingredient-free adhesive layer

| | | |
|---|---|---|
| Paraffin, viscous | 2.40 | 0.66 |
| BIO-PSA ® SRS7-4501 | 24.40 | 9.85 |
| (e.g., 68% solid content) | | |
| BIO-PSA ® SRS7-4601 | 73.20 | 31.20 |
| (e.g., 65% solid content) | | |
| n-Heptane | — | 0.59 |
| Sum | 100.0 | 42.3 |

First, the matrix layer (2) was prepared. For that, an aqueous 1% (w/w %) sodium metabisulfite solution as well as an ethanolic 25% (w/w %) PVP K90 solution were prepared. The corresponding amount of sodium metabisulfite solution was added to the corresponding amount of PVP solution in a suitable glass vessel and stirred for ca. 30 min. Ascorbyl palmitate and tocopherol were added and stirred. Then, the active ingredient rotigotine was slowly added under stirring and heating in a water bath and stirred at approximately 60° C. until complete dissolution. After the mass had cooled down again the silicone adhesives were sequentially added and briefly stirred. Then, the balancing solvent heptane was added and stirred. The coating mass was stirred until all was visually homogeneously distributed. Subsequently, the coating mass was spread out on a fluorine-coated foil, e.g., Scotchpak™ 9709, as a thin film and subsequently heated in a drying tunnel of ca. 52 cm in length with 4 separate sections each at ca. 45, 60, 80, 99° C. with a rate of ca. 0.16 m/min, so that the solvents were almost completely removed. The dried matrix was about 50 g/m² and was laminated with a release liner, e.g., of polyethylene terephthalate (PET) in a thickness of 19 μm.

For the preparation of the initially active ingredient-free adhesive layer (3) the silicone adhesives were added into a suitable glass vessel and briefly stirred. Then, paraffin and the balancing solvent heptane were sequentially added and stirred. The coating mass was stirred until all was visually homogeneously distributed. Thereafter, the coating mass was spread out on a fluorine-coated foil, e.g., Scotchpak™ 9709, as a thin film and subsequently heated for e.g., 5 min. at 85° C., so that the solvents were almost completely removed. The dried initially active ingredient-free adhesive layer (3) had a matrix weight in the range of from 25-30 g/m² and was laminated with the matrix layer onto the open matrix of the initially active ingredient-free adhesive layer (3) while peeling off its release liner.

WpUA [g/m²]: weight per unit area

PVP K90: polyvinyl pyrrolidone K-90 (BASF SE)

rh: relative humidity

RSD: relative standard deviation

RS: matrix layer

HS: initially active ingredient-free adhesive layer

mon: months

RT: room temperature

The Thus Prepared Transdermal Therapeutic Systems for Example have the Following General Composition:

Monolayer formulations with an active ingredient-containing matrix layer comprising rotigotine and PVP K90 in certain weight ratios and weight percentages based on the total weight of the matrix layer; 0-3% by weight of viscous paraffin; various combinations of one or two non-silanol-reduced or silanol-reduced non-amine-resistant silicone adhesives (Dow Corning®); at least one antioxidant, e.g., tocopherol 0.05-0.1% by weight, ascorbyl palmitate 0.02-0.1% by weight and Na metabisulfite 0.0006-0.0021% by weight; and a matrix weight of ca. 50-60 g/m².

Bilayer formulations with an active ingredient-containing matrix layer comprising rotigotine and PVP K90 in certain weight ratios and weight percentages based on the total weight of the matrix layer; non-silanol-reduced or silanol-reduced non-amine-resistant silicone adhesives BIO-PSA 7-4501 or BIO-PSA SRS7-4501 (Dow Corning®); at least one antioxidant, e.g., tocopherol 0.05-0.1% by weight, and/or ascorbyl palmitate 0.02-0.1% by weight and 0.0006-0.0021% by weight of Na metabisulfite; a matrix weight of ca. 2550 g/m²; and an additional initially active ingredient-free pressure sensitive adhesive layer (3) comprising various combinations of one or two non-silanol-free non-amine-resistant silicone adhesives (Dow Corning®) and 0-3% by weight of viscous paraffin.

The Following Specific Monolayer and Bilayer Formulations have been Prepared:
A) Monolayer Formulations with Different Mixing Ratios of Non-Amine-Resistant Silicone Adhesives without Paraffin

Various monolayer formulations without paraffin with different mixing ratios of BIO-PSA SRS7-4501 to BIO-PSA SRS7-4601 and BIO-PSA 7-4501 to BIO-PSA 7-4601 in the matrix layer were prepared. For these monolayer formulations various combinations of the backing layers Scotchpak 1109 (3M Corporation) and Hostaphan® MN19 (Mitsubishi Polyester Film) and the release liners Scotchpak 1022, Scotchpak 9744 and Scotchpak 9709 (3M Corporation) have been used. The resulting TTS were stored at 25° C. and 40° C. over various periods and the separation force, adhesion strength and tack were investigated. Additionally, in vitro permeation on human heat separated epidermis of some formulations was determined.

The separation force can be determined as the force required to detach a sample from its release liner in a specified angle and at a defined rate. To determine it, TDS of a defined size, e.g., 10 cm², are blanked and conditioned at 23±1° C. and 50±5% rh. A guiding strip having the width of the TDS is attached to the TDS. Then, the TDS with the release liner down is fixed on a tool carriage by means of double-sided adhesive tape. This is placed in a tensile tester, e.g., Texture Analyser plus by Stable Micro Systems, such that the TDS is peeled off in an angle of 90°. In general, the measurement takes place with a rate of 300±30 mm/min typically at 23±1° C. and 50±5% rh. The separation force is the mean force standardized to a sample width of 25 mm [N/25 mm] and measured over the separation path.

The adhesion strength can be determined as the force required to detach a sample from a suitable carrier in a specified angle and at a defined rate. To determine the adhesion strength TDS of a defined size, e.g., 10 cm², are blanked and conditioned at 23±1° C. and 50±5% rh. A guiding strip, e.g., made of double-sided adhesive tape, having the width of the TDS is attached. The TDS is adhered to a test panel, e.g., made of steel, while peeling off its release liner and thereafter pressed on for e.g., 1 min with a 2 kg weight while lying between two glass plates. The test panel is vertically attached in a tensile tester, e.g., Texture Analyser plus by Stable Micro Systems, and the guiding strip is clamped such that the TDS is peeled off in a 90° angle. In general, the measurement takes place with a specified rate of 300±30 mm/min typically at 23±1° C. and 50±5% rh. The adhesion strength is the mean force standardized to a sample width of 25 mm [N/25 mm] and measured over the path.

Tack can be determined as the maximum force required to completely separate a stainless-steel test piece from the adhesive layer of a TDS. To determine the tack a plaster or laminate is conditioned at 23±1° C. and 50±5% rh and thereafter fixed on a perforated support plate with the open adhesive matrix while peeling off its release liner. The plate is attached in a tensile tester, e.g., Texture Analyser plus by Stable Micro Systems. Typically at 23±1° C. and 50±5% rh the test piece is pressed onto the top of the sample and peeled off after a defined contact time of generally 2 sec. A series of measurements after having peeled off the release liner from the first sample should be completed within 30 min. The maximum force (tack; [N]) to separate the bond between test piece and adhesive layer is established.

TABLE 1
Monolayer formulations without paraffin

| | | | | |
|---|---|---|---|---|
| | | Polymer | WpUA | Release Liner/ |
| Batch | Composition of the matrix | Ratio | [g/m²] | Backing Layer |

Batches with silanol-reduced (SRS) non-amine-resistant
silicone adhesives BIO-PSA SRS7-4501 & SRS7-4601

| | | | | |
|---|---|---|---|---|
| 538ROTTDS | rotigotine 9%, PVP K90 7%, | 1:1 | 50 | Scotchpak 1022, |
| | tocopherol 0.05%, | | | Scotchpak 1109 |
| | BIO-PSA SRS7-4501 41.975%, | | | |
| | BIO-PSA SRS7-4601 41.975% | | | |
| 536ROTDS | rotigotine 9%, PVP K90 7%, | 1:1.5 | 50 | Scotchpak 1022, |
| | tocopherol 0.05%, | | | Scotchpak 1109 |
| | BIO-PSA SRS7-4501 33.58%, | | | |
| | BIO-PSA SRS7-4601 50.37% | | | |
| 537ROTDS | rotigotine 9%, PVP K90 7%, | 1:2 | 50 | Scotchpak 9744, |
| | tocopherol 0.05%, | | | Scotchpak 1109 |
| | BIO-PSA SRS7-4501 27.98%, | | | |
| | BIO-PSA SRS7-4601 55.97% | | | |
| 539ROTTDS | rotigotine 9%, PVP K90 7%, | 1:3 | 50 | Scotchpak 1022, |
| | tocopherol 0.05%, | | | Scotchpak 1109 |
| | BIO-PSA SRS7-4501 20.99%, | | | |
| | BIO-PSA SRS7-4601 62.96% | | | |
| 534ROTTDS | rotigotine 0%, PVP K90 7.7%, | 1:1 | 45.5 | Scotchpak 9744, |
| | tocopherol 0.05%, | | | Scotchpak 1109 |
| | BIO-PSA SRS7-4501 46.13%, | | | |
| | BIO-PSA SRS7-4601 46.13% | | | |
| Neupro | rotigotine 9%, PVP K90 2%, | 1:1 | 50 | Scotchpak 9744, |
| according to | tocopherol 0.05%, | | | Scotchpak 1109 |
| EP1524975 | ascorbyl palmitate 0.02%, | | | |
| | Na metabisulfite 0.0006%, | | | |
| | BIO-PSA 7-4201 44.465%, | | | |
| | BIO-PSA 7-4301 44.465% | | | |
| Neupro | rotigotine 7.5%, PVP K90 3.33%, | 1:1 or 1:2 | 60 | Scotchpak 9744, |
| according to | tocopherol 0.05%, | | | Scotchpak 1109 |
| EP2515887 | ascorbyl palmitate 0.02%, | | | |
| | Na metabisulfite 0.0006%, | | | |
| | BIO-PSA 7-4201 35.64-44.55%, | | | |
| | BIO-PSA 7-4301 44.55-53.46% | | | |
| 576ROTTDS | rotigotine 9%, PVP K90 7%, | 0:1 | 50 | Scotchpak 9709, |
| | tocopherol 0.05%, | | | Scotchpak 1109 |
| | ascorbyl palmitate 0.02%, | | | |
| | Na metabisulfite 0.00006% | | | |
| | BIO-PSA SRS7-4601 83.929% | | | |
| 564ROTTDS | rotigotine 9%, PVP K90 7%, | 0:1 | 50 | Eastmen, |
| | tocopherol 0.05%, | | | Scotchpak 1109 |
| | ascorbyl palmitate 0.02%, | | | |
| | Na metabisulfite 0.00006% | | | |
| | BIO-PSA SRS7-4601 83.929% | | | |
| 588ROTTDS | rotigotine 9%, PVP K90 7%, | 1:3 | 50 | Scotchpak 9709, |
| | tocopherol 0.05%, | | | Scotchpak 1109 |
| | ascorbyl palmitate 0.02%, | | | |
| | Na metabisulfite 0.00021% | | | |
| | BIO-PSA SRS7-4501 20.982%, | | | |
| | BIO-PSA SRS7-4601 62.946% | | | |
| 550ROTTDS | rotigotine 9%, PVP K90 7%, | 1:1 | 50 | Scotchpak 9709, |
| | tocopherol 0.05%, | | | Scotchpak 1109 |
| | ascorbyl palmitate 0.02%, | | | |
| | Na metabisulfite 0.00006% | | | |
| | BIO-PSA SRS7-4501 41.965%, | | | |
| | BIO-PSA SRS7-4601 41.965% | | | |
| 589ROTTDS | rotigotine 9%, PVP K90 7%, | 3:1 | 50 | Scotchpak 9709, |
| | tocopherol 0.05%, | | | Scotchpak 1109 |
| | ascorbyl palmitate 0.02%, | | | |
| | Na metabisulfite 0.00021% | | | |
| | BIO-PSA SRS7-4501 62.946%, | | | |
| | BIO-PSA SRS7-4601 20.982% | | | |
| 577ROTTDS | rotigotine 9%, PVP K90 7%, | 1:0 | 50 | Scotchpak 9709, |
| | tocopherol 0.05%, | | | Scotchpak 1109 |
| | ascorbyl palmitate 0.02%, | | | |
| | Na metabisulfite 0.00021% | | | |
| | BIO-PSA SRS7-4501 83.928% | | | |

Batches with non-silanol-reduced non-amine-resistant
silicone adhesives BIO-PSA 7-4501 & 7-4601

| | | | | |
|---|---|---|---|---|
| 583ROTTDS | rotigotine 9%, PVP K90 7%, | 0:1 | 50 | Scotchpak 9709, |
| | tocopherol 0.05%, | | | Scotchpak 1109 |
| | ascorbyl palmitate 0.02%, | | | |
| | Na metabisulfite 0.00021% | | | |
| | BIO-PSA 7-4601 83.929% | | | |
| 586ROTTDS | rotigotine 9%, PVP K90 7%, | 1:3 | 50 | Scotchpak 9709, |
| | tocopherol 0.05%, | | | Scotchpak 1109 |
| | ascorbyl palmitate 0.02%, | | | |
| | Na metabisulfite 0.00021% | | | |
| | BIO-PSA SRS7-4501 20.982%, | | | |
| | BIO-PSA SRS7-4601 62.946% | | | |
| 581ROTTDS | rotigotine 9%, PVP K90 7%, | 1:1 | 50 | Scotchpak 9709, |
| | tocopherol 0.05%, | | | Scotchpak 1109 |
| | ascorbyl palmitate 0.02%, | | | |
| | Na metabisulfite 0.00021% | | | |
| | BIO-PSA SRS7-4501 41.964%, | | | |
| | BIO-PSA SRS7-4601 41.964% | | | |
| 587ROTTDS | rotigotine 9%, PVP K90 7%, | 3:1 | 50 | Scotchpak 9709, |
| | tocopherol 0.05%, | | | Scotchpak 1109 |
| | ascorbyl palmitate 0.02%, | | | |
| | Na metabisulfite 0.00021% | | | |
| | BIO-PSA SRS7-4501 62.946%, | | | |
| | BIO-PSA SRS7-4601 20.982% | | | |
| 582ROTTDS | rotigotine 9%, PVP K90 7%, | 1:0 | 50 | Scotchpak 9709, |
| | tocopherol 0.05%, | | | Scotchpak 1109 |
| | ascorbyl palmitate 0.02%, | | | |
| | Na metabisulfite 0.00021% | | | |
| | BIO-PSA SRS7-4501 83.928% | | | |

B) Monolayer Formulations with Different Mixing Ratios of Non-Amine-Resistant Silicone Adhesives with Paraffin

Various monolayer formulations have been prepared in analogy to A) with a small amount of paraffin with different mixing ratios of BIO-PSA SRS7-4501 to BIO-PSA SRS7-4601 and BIO-PSA 7-4501 to BIO-PSA 7-4601 (Dow Corning®) in the matrix layer. For these monolayer formulations various combinations of the backing layers Scotchpak 1109 (3M Corporation) and Hostaphan® MN19 (Mitsubishi Polyester Film) and the release liners Scotchpak 9744 and Scotchpak 9709 (3M Corporation) have been used. The resulting TTS were stored at 25° C. and 40° C. over various periods and the separation force, adhesion strength and tack were investigated. Additionally, in vitro permeation on human heat separated epidermis of some formulations was determined.

TABLE 2
Monolayer formulations with paraffin

| | | | | |
|---|---|---|---|---|
| | | Polymer | WpUA | Release Liner/ |
| Batch | Composition of the matrix | Ratio | [g/m²] | Backing Layer |

Batches with silanol-reduced (SRS) non-amine-resistant silicone adhesives BIO-PSA
SRS7-4501 & SRS7-4601 or non-silanol-reduced non-amine-resistant silicone
adhesives BIO-PSA 7-4501 & 7-4601 with a small addition of viscous paraffin

| | | | | |
|---|---|---|---|---|
| 574ROTTDS | rotigotine 9%, PVP K90 7%, | 0:1 | 50 | Scotchpak 9744, |
| | tocopherol 0.05%, | | | Scotchpak 1109 |
| | ascorbyl palmitate 0.02% | | | |
| | Na metabisulfite 0.00006%, | | | |
| | paraffin 2%, | | | |
| | BIO-PSA SRS7-4501 82% | | | |
| 599ROTTDS | rotigotine 9%, PVP K90 7%, | 0:1 | 50 | Scotchpak 9709, |
| | tocopherol 0.05%, | | | Scotchpak 1109 |
| | ascorbyl palmitate 0.02%, | | | |
| | Na metabisulfite 0.00021%, | | | |
| | paraffin 1%, | | | |
| | BIO-PSA SRS7-4501 82.93% | | | |
| 611ROTTDS | rotigotine 9%, PVP K90 7%, | 1:3 | 50 | Scotchpak 9709, |
| | tocopherol 0.1% | | | Hostapahn MN19 |
| | Na metabisulfite 0.0021%, | | | |
| | paraffin 2%, | | | |
| | BIO-PSA 7-4501 20.474%, | | | |
| | BIO- PSA 7-4601 61.423% | | | |
| 602ROTTDS | rotigotine 9%, PVP K90 7%, | 1:3 | 50 | Scotchpak 9709 |
| | tocopherol 0.1%, | | | Hostaphan MN19 |
| | Na metabisulfite 0.0021%, | | | |
| | paraffin 1%, | | | |
| | BIO-PSA 7-4501 20.724%, | | | |
| | BIO-PSA 7-4601 62.173% | | | |
| 614ROTTDS | rotigotine 9%, PVP K90 7%, | 0:1 | 50 | Scotchpak 9709 |
| | tocopherol 0.1%, | | | Hostaphan MN19 |
| | Na metabisulfite 0.0021%, | | | |
| | paraffin 2%, | | | |
| | BIO-PSA 7-4601 81.8979% | | | |
| 616ROTTDS | rotigotine 7.5%, PVP K90 5.83%, | 1:3 | 60 | Scotchpak 9709 |
| | tocopherol 0.05%, | | | Hostaphan MN19 |
| | Na metabisulfite 0.0021%, | | | |
| | ascorbyl palmitate 0.02%, | | | |
| | paraffin 2.1%, | | | |
| | BIO-PSA SRS7-4501 21.12%, | | | |
| | BIO-PSA SRS7-4601 63.37% | | | |
| 616ROTTDS | rotigotine 7.5%, PVP K90 5.83%, | 1:3 | 50 | Scotchpak 9709 |
| | tocopherol 0.05%, | | | Hostaphan MN19 |
| | Na metabisulfite 0.0021%, | | | |
| | ascorbyl palmitate 0.02%, | | | |
| | paraffin 2.1%, | | | |
| | BIO-PSA SRS7-4501 21.12%, | | | |
| | BIO-PSA SRS7-4601 63.37% | | | |

C) Bilayer Formulations with Different Mixing Ratios of Non-Amine-Resistant Silicone Adhesives with and without Paraffin

Various bilayer formulations with different amounts of rotigotine and BIO-PSA SRS7-4501 or BIO-PSA 7-4501 (Dow Corning®) in the matrix layer have been prepared. The bilayer formulations additionally had an additional initially active ingredient-free pressure sensitive adhesive layer on the side of the matrix layer opposite to the side contacting the backing layer. The additional initially active ingredient-free pressure sensitive adhesive layer was formulated with or without a small amount of paraffin and with amounts of BIO-PSA SRS7-4501 and/or BIO-PSA SRS7-4601 or BIO-PSA 7-4501 (Dow Corning®).

Here, the ratio of silicone adhesive of medium tack and silicone adhesive of high tack had to be selected such that both good adhesion strength and tack and a cold flow as low as possible and sufficiently high cohesion resulted.

Various combinations of the backing layers Scotchpak 1109 (3M Corporation) and Hostaphan® MN19 (Mitsubishi Polyester Film) and the release liners Scotchpak 9744, Scotchpak 1022 and Scotchpak 9709 (3M Corporation) and Primeliner 100 μm 78BT, Primeliner 75 μm 78HL (Loparex International B.V.) have been used. The resulting TTS were stored at 25° C. and 40° C. over different periods of time and separation force, adhesion strength and tack were investigated. Additionally, in vitro permeation on human heat separated epidermis of some formulations was determined.

In the bilayer formulations the effect of an additional initially active ingredient-free pressure sensitive adhesive layer on separation force, adhesion strength, tack and in vitro permeation should be investigated.

TABLE 3
Bilayer formulations with and without paraffin

| | | | WpUA Matrix/ | |
|---|---|---|---|---|
| | | Composition of the | additional active | |
| | | additional initially | ingredient-free | |
| | | active ingredient- free | pressure sensitive | |
| | | pressure sensitive | adhesive layer | Release Liner/ |
| Batch | Composition of the matrix layer | adhesive layer | [g/m²] | Backing Layer |
| 569_568ROTTDS | rotigotine 15%, PVP K90 8.5%, | Duro-Tak 87-4098 100% | 30/30 | Primeliner ™ |
| | BIO-PSA SRS7-4501 76.5% | | | 100 μm 78BT/ |
| | | | | Scotchpak 1109 |
| 570_568ROTTDS | rotigotine 15%, PVP K90 8.5%, | Duro-Tak 87-626A 53.8%, | 30/30 | Primeliner ™ |
| | BIO-PSA SRS7-4501 76.5% | Duro-Tak 87-625A 23.1%, | | 100 μm 78BT/ |
| | | Indopol H-1900 23.1% | | Scotchpak 1109 |
| 571_568ROTTDS | rotigotine 15%, PVP K90 8.5%, | BIO-PSA SRS7-4601 100% | 30/30 | Scotchpak 9744, |
| | BIO-PSA SRS7-4501 76.5% | | | Scotchpak 1109 |
| 572_568ROTTDS | rotigotine 15%, PVP K90 8.5%, | SBS 45%, paraffin 20%, | 30/30 | Primeliner ™ |
| | BIO-PSA SRS7-4501 76.5 | saturated alicyclic resin 35% | | 100 μm 78BT/ |
| | | | | Scotchpak 1109 |
| 592_591ROTTDS | rotigotine 9%, PVP K90 7%, | BIO-PSA SRS7-4601 100% | 50/30 | Scotchpak 9709, |
| | tocopherol 0.05%, | | | Scotchpak 1109 |
| | Na metabisulfite 0.00021%, | | | |
| | ascorbyl palmitate 0.02%, | | | |
| | BIO-PSA SRS7-4501 83.928% | | | |
| 593_590ROTTDS | rotigotine 9%, PVP K90 7%, | BIO-PSA 7-4601 100% | 50/30 | Scotchpak 9709, |
| | tocopherol 0.05%, | | | Hostaphan MN15 |
| | Na metabisulfite 0.00021%, | | | |
| | ascorbyl palmitate 0.02%, | | | |
| | BIO-PSA 7-4501 83.928% | | | |
| 612_613ROTTDS | rotigotine 11.25%, PVP K90 8.75%, | BIO-PSA SRS7-4601 100% | 40/27.5 | Scotchpak 9709 |
| | tocopherol 0.1%, | | | Hostaphan MN19 |
| | Na metabisulfite 0.0021%, | | | |
| | BIO-PSA SRS7-4501 79.898% | | | |
| 618_617ROTTDS | rotigotine 9%, PVP K90 7%, | BIO-PSA SRS7-4501 24.4%, | 50/27.5 | Scotchpak 9709 |
| | tocopherol 0.05%, | BIO-PSA SRS7-4601 73.2%, | | Hostaphan MN19 |
| | Na metabisulfite 0.0021%, | paraffin 2.4% | | |
| | ascorbyl palmitate 0.02%, | | | |
| | BIO-PSA SRS7-4501 83.93% | | | |

D) Monolayer Formulations with Non-Amine-Resistant Silicone Adhesives and Different Concentrations of Polyvinyl Pyrrolidone

Various monolayer formulations with different amounts of PVP K90 with a fixed amount of 9% by weight of rotigotine, based on the total weight of the matrix layer, and BIO-PSA SRS7-4501 to BIO-PSA SRS7-4601 (Dow Corning®) in a mixing ratio of 1:1 in the matrix layer have been prepared.

Backing layer Scotchpak 1109 (3M Corporation) and release liner Scotchpak 9744 have been used. The resulting TTS were stored at 25° C. and 40° C. over different periods of time and the appearance was investigated. Additionally, in vitro permeation on human heat separated epidermis (HSE) of some formulations was determined. It was the aim to investigate the effect of the amount of PVP K90 on the recrystallization of rotigotine and in vitro permeation.

TABLE 4
Monolayer formulations with different amounts of PVP K90
with a constant amount of 9% by weight of rotigotine.

| | | Polymer | WpUA | Release Liner/ |
|---|---|---|---|---|
| Batch | Composition of the matrix layer | Ratio | [g/m²] | Backing Layer |
| 506ROTTDS | rotigotine 9%, PVP K90 3.2%, | 1:1 | 50 | Scotchpak 9744, |
| | tocopherol 0.05%, | | | Scotchpak 1109 |
| | BIO-PSA SRS7-4501 43.875%, | | | |
| | BIO-PSA SRS7-4601 43.875% | | | |
| 525ROTTDS | rotigotine 9%, PVP K90 5%, | 1:1 | 50 | Scotchpak 9744, |
| | tocopherol 0.05% | | | Scotchpak 1109 |
| | BIO-PSA SRS7-4501 42.975%, | | | |
| | BIO-PSA SRS7-4601 42.975% | | | |
| 526ROTTDS | rotigotine 9%, PVP K90 6%, | 1:1 | 50 | Scotchpak 9744, |
| | tocopherol 0.05%, | | | Scotchpak 1109 |
| | BIO-PSA SRS7-4501 42.475%, | | | |
| | BIO-PSA SRS7-4601 42.475% | | | |
| 511ROTTDS | rotigotine 9%, PVP K90 7%, | 1:1 | 50 | Scotchpak 9744, |
| | tocopherol 0.05%, | | | Scotchpak 1109 |
| | BIO-PSA SRS7-4501 41.975%, | | | |
| | BIO-PSA SRS7-4601 41.975% | | | |
| 513ROTTDS | rotigotine 9%, PVP K90 8%, | 1:1 | 50 | Scotchpak 9744, |
| | tocopherol 0.05%, | | | Scotchpak 1109 |
| | BIO-PSA SRS7-4501 41.475%, | | | |
| | BIO-PSA SRS7-4601 41.475% | | | |
| 554ROTTDS | rotigotine 9%, PVP K90 9%, | 1:1 | 50 | Scotchpak 9744, |
| | tocopherol 0.05%, | | | Scotchpak 1109 |
| | BIO-PSA SRS7-4501 40.975%, | | | |
| | BIO-PSA SRS7-4601 40.975% | | | |

### Example 2 (Separation Force, Adhesion Strength and Tack)

It was the aim to investigate the effect of the mixing ratios of the non-amine-resistant silicone adhesives BIO-PSA SRS7-4501 to BIO-PSA SRS7-4601 and BIO-PSA 7-4501 to BIO-PSA 7-4601 (Dow Corning®) on separation force, adhesion strength and tack to thereby be able to set an optimum mixing ratio for the matrix layer. It was the aim also to investigate the effect of paraffin on separation force, adhesion strength and tack.

### A) Separation Force

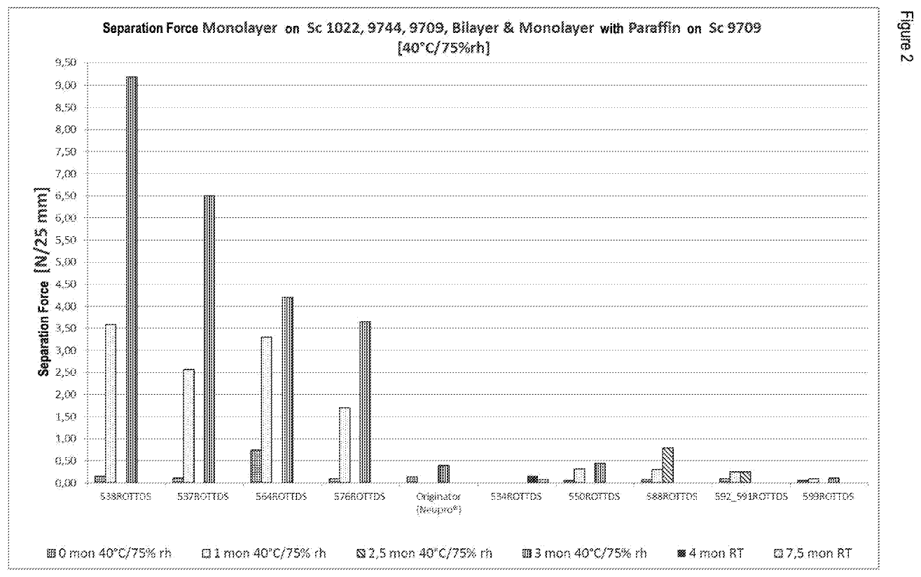

As can be clearly seen in table 5 and FIG. 2 TTS formulations with rotigotine in combination with one or more silicone adhesives with mainly free silanol groups (non-amine-resistant silicone adhesives) in the matrix layer of monolayer formulations and a release liner coated with fluoropolymer, such as e.g., Scotchpak 1022 or Scotchpak 9744 (3M Corporation) during storage resulted in a large increase in the separation force. In case of the placebo formulation without rotigotine when using silicone adhesives with still free silanol groups and the same release liner types no increase in the separation force was observed.

A release liner coated with fluorosilicone, such as e.g., Scotchpak 9709 (3M Corporation) with the same or similar formulations showed a significantly lower increase in the separation force when using rotigotine and one or more silicone adhesives with still free silanol groups (cf., FIG. 2 and Table 5).

TABLE 5
Separation force of silicone adhesive formulations with non-silanol-reduced silicone adhesives
and with silanol-reduced non-amine-resistant silicone adhesives in different polymer adhesive
mixing ratios with or without paraffin during storage over 0-3 months at 40° C./75% rh.

| | Separation | Separation force | Separation force | Separation | Separation |
|---|---|---|---|---|---|
| | force after | after 1 mon at | after 3 mon at | force after 4 | force after 7.5 |
| Batch 1 Release Liner | 0 mon | 40° C./75% (rh) | 40° C./75% rh | mon at RT | mon at RT |
| 538ROTTDS/ | 0.15 | 3.58 | 9.18 | ./. | ./. |
| Scotchpak ™ 1022 | (48% RSD) | (16% RSD) | (8% RSD) | | |
| 537ROTTDS/ | 0.10 | 2.56 | 6.51 | ./. | ./. |
| Scotchpak ™ 9744 | (36% RSD) | (8% RSD) | (8% RSD) | | |
| 564ROTTDS/ | 0.74 | 3.3 | 4.2 | ./. | ./. |
| Eastmen Release | (8% RSD) | (12% RSD) | (8% RSD) | | |
| Liner | | | | | |
| 576ROTTDS/ | 0.09 | 1.70 | 3.65 | ./. | ./. |
| Scotchpak ™ 9709 | (35% RSD) | (2% RSD) | 11% RSD) | | |
| Neupro ® lot: | 0.125 | ./. | 0.389 | ./. | ./. |
| 56659203/ | (29% RSD) | | (27% RSD) | | |
| Scotchpak ™ 9744 | | | | | |
| 534ROTTDS/ | ./. | 0.05 | 0.08 | 0.16 | 0.07 |
| Scotchpak ™ 9744 | | (54% RSD) | (34% RSD) | (54% RSD) | (40% RSD) |
| 550ROTTDS/ | 0.05 | 0.31 | 0.44 | ./. | ./. |
| Scotchpak ™ 9709 | (42% RSD) | (37% RSD) | (14% RSD) | | |
| 588ROTTDS/ | 0.06 | 0.30 | 0.79 | ./. | ./. |
| Scotchpak ™ 9709 | (9% RSD) | (17% RSD) | (25% RSD) | | |
| | | | (2.5 mon) | | |
| 592_591ROTTDS/ | 0.09 | 0.25 | 0.25 | ./. | ./. |
| Scotchpak ™ 9709 | (31% RSD) | (24% RSD) | (9% RSD) | | |
| | | | 2.5 mon | | |
| 618_617ROTTDS/ | 0.07 | 0.14 | ./. | ./. | ./. |
| Scotchpak ™ 9709 | (54% RSD) | (31% RSD) | | | |
| 599ROTTDS/ | 0.05 | 0.09 | 0.14 | ./. | ./. |
| Scotchpak ™ 9709 | (15% RSD) | (29% RSD) | (16% RSD) | | |
| 602ROTTDS/ | 0.07 | 0.20 | 0.39 | ./. | ./. |
| Scotchpak ™ 9709 | (58% RSD) | (28% RSD) | (23% RSD) | | |
| 611 ROTTDS/ | 0.24 | 0.37 | 0.46 | ./. | ./. |
| Scotchpak ™ 9709 | (39% RSD) | (22% RSD) | (34% RSD) | | |
| 614ROTTDS/ | 0.62 | 0.45 | ./. | ./. | ./. |
| Scotchpak ™ 9709 | (9% RSD) | (11% RSD) | | | |
| 616ROTTDS/ | 0.15 | ./. | ./. | ./. | ./. |
| Scotchpak ™ 9709 | (10% RSD) | | | | |
| 583ROTTDS/ | 0.17 | 0.21 | 0.95 | ./. | ./. |
| Scotchpak ™ 9709 | (38% RSD) | (13% RSD) | (51% RSD) | | |
| 587ROTTDS/ | 0.04 | 0.09 | 0.16 | ./. | ./. |
| Scotchpak ™ 9709 | (28% RSD) | (39% RSD) | (16% RSD) | | |
| 581ROTTDS/ | 0.10 | 0.48 | 0.15 | ./. | ./. |
| Scotchpak ™ 9709 | (44% RSD) | (21% RSD) | (29% RSD) | | |

Surprisingly, also formulations with rotigotine and mixtures of the silanol-reduced non-amine-resistant silicone adhesives BIO-PSA SRS7-4501 and BIO-PSA SRS7-4601 (Dow Corning®) in contrast to rotigotine with the silanol-reduced non-amine-resistant silicone adhesive BIO-PSA SRS7-4601 (Dow Corning®) alone had a lower increase in the separation force. The non-silanol-reduced non-amine-resistant silicone adhesives BIO-PSA 7-4501 and BIO-PSA 7-4601 (Dow Corning®) also showed a lower increase in the separation force compared with the non-silanol-reduced non-amine-resistant silicone adhesive BIO-PSA 7-4601 (Dow Corning®) alone.

### B) Adhesion Strength

Additionally, the adhesion strength of various TTS formulations comprising rotigotine and different non-amine resistant silicone adhesives and adhesive mixtures has been compared with a placebo formulation and the Neupro® TTS available on the market after storage over 0 to 3 months.

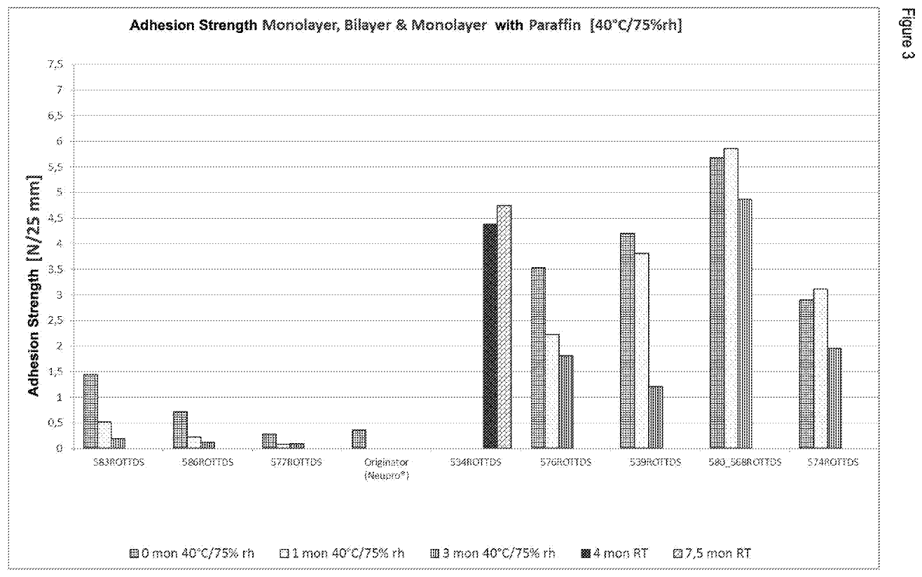

As can clearly be seen in Table 6 and FIG. 3 TTS formulations with rotigotine in combination with one or more non-silanol-reduced silicone adhesives (BIO-PSA 7-4501 and BIO-PSA 7-4601; Dow Corning®) (non-silanol-reduced non-amine-resistant silicone adhesives) resulted in a relatively low adhesion strength. Said relatively low adhesion strength even decreased strongly with storage times of 1 and 3 month(s) at 40° C./75% rh, so that after 1 month of storage it already resulted in a lower adhesion strength than the product Neupro®. With the placebo formulation there was no decrease in the adhesion strength despite of the use of non-amine-resistant silicone adhesives.

Surprisingly, formulations of silanol-reduced non-amine-resistant silicone adhesives (BIO-PSA SRS7-4501 and BIO-PSA SRS7-4601 by Dow Corning®) with rotigotine, even though these have less free silanol groups and thus can interact less with surfaces than the non-silanol-reduced silicone adhesives, showed a significantly higher adhesion strength and a lower relevant decrease in adhesion strength too, so that the absolute adhesion strength value after 0 to 3 months of storage at 40° C./75% rh was still higher than in case of the Neupro® product.

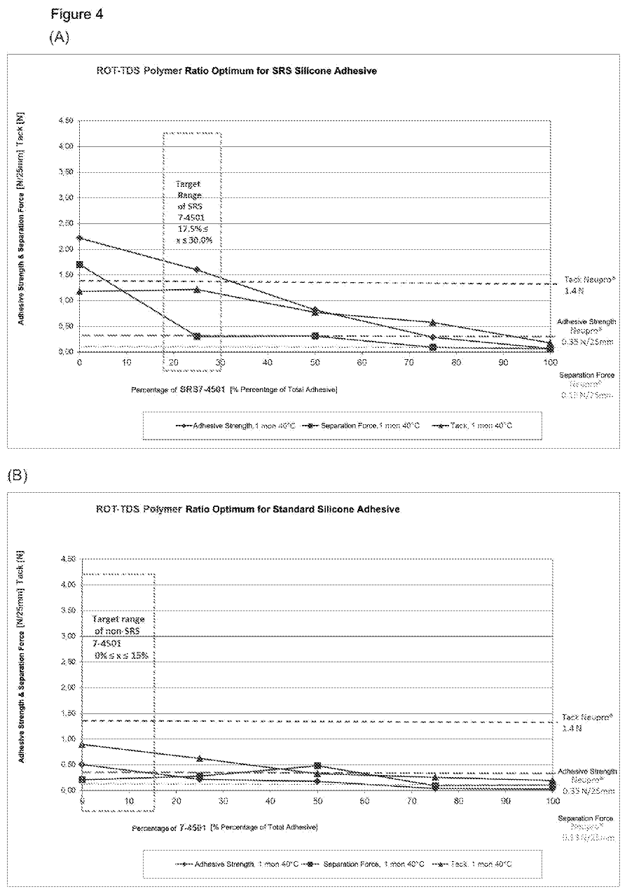

Moreover, formulations with silicone adhesive ratios of ca. 17.5-30.0% by weight BIO-PSA SRS7-4501:82.5-70.0% by weight BIO-PSA SRS7-4601 with respect to the total percentage of the silicone adhesives BIO-PSA SRS7-4501 and BIO-PSA SRS7-4601 had a high adhesion strength and did not result in a relevant increase in separation force (cf., FIG. 4 and Table 6) compared with formulations only with BIO-PSA SRS7-4601 (Dow Corning®) (cf., FIG. 3).

Surprisingly, addition of paraffin in low amounts of ca. 1-3% by weight to the formulations resulted in a significantly higher adhesion strength which had a slightly lower decrease during the storage over 1-3 month(s) (cf., FIG. 3 and Table 6).

The presence of paraffin according to the invention therefore also allows to use non-silanol-reduced non-amine-resistant silicone adhesives. The adhesion strength with these silicone adhesives is improved over the Neupro® product, even though not quite as strong as when using silanol-reduced non-amine-resistant silicone adhesives, which are therefore preferred.

TABLE 6
Adhesion strength of silicone adhesive formulations with non-silanol-reduced silicone
adhesives and with silanol-reduced silicone adhesives in different polymer adhesive mixing
ratios with or without paraffin during storage over 0-3 month(s) at 40° C./75% rh.

| | | | Adhesion | Adhesion | | |
|---|---|---|---|---|---|---|
| | | | Strength | Strength | Adhesion | Adhesion |
| | Adhesive | Adhesion | after 1 mon | after 3 mon | Strength | Strength |
| | Type/Mixing | Strength | at 40^{o} C./ | at 40^{o} C./ | after 4 mon | after 7.5 |
| Batch | Ratio | after 0 mon | 75% rh | 75% rh | at RT | mon at RT |
| 583ROTTDS | 7-4601 | 1.44 | 0.51 | 0.19 | | |
| | | (7% RSD) | (12% RSD) | (45% RSD) | | |
| 586ROTTDS | 7-4501:4601/ | 0.71 | 0.22 | 0.12 | | |
| | 1:3 | (10% RSD) | (67% RSD) | (48% RSD) | | |
| 577ROTTDS | SRS7-4501 | 0.28 | 0.07 | 0.09 | | |
| | | (24% RSD) | (55% RSD) | (28% RSD) | | |
| Neupro ® lot: | | 0.35 | ./. | ./. | ./. | ./. |
| 55338204 | | (56% RSD) | | | | |
| 534ROTTDS | SRS7- | ./. | 4,20 | 3.82 | 4.37 | 4.74 |
| | 4501:4601/ | | (7% RSD) | (2% RSD) | (2% RSD) | (4% RSD) |
| | 1:1 | | | | | |
| 576ROTTDS | SRS7-4601 | 3.53 | 2.22 | 1.81 | ./. | ./. |
| | | (7% RSD) | (21% RSD) | 29% RSD) | | |
| 539ROTTDS | SRS7- | 4.21 | 3.76 | 1.18 | ./. | ./. |
| | 4501:4601/ | (3% RSD) | (20% RSD) | (31% RSD) | | |
| | 1:3 | | | | | |
| 571_568ROTTDS | RS: SRS7- | 5.68 | 5.68 | 4.86 | ./. | ./. |
| | 4501 | (5% RSD) | (3% RSD) | (4% RSD) | | |
| | HS: SRS7- | | | | | |
| | 4601 | | | | | |
| 592_591ROTTDS | RS: SRS7- | 3.52 | 2.23 | 0.79 | ./. | ./. |
| | 4501 | (14% RSD) | (27% RSD) | (8% RSD) | | |
| | HS: SRS7- | | | | | |
| | 4601 | | | | | |
| 618_617ROTTDS | RS: SRS7- | 6.65 | 4.60 | ./. | ./. | ./. |
| | 4501 | (7% RSD) | (25% RSD) | | | |
| | HS: | | | | | |
| | SRS7- | | | | | |
| | 4501:SRS7- | | | | | |
| | 4601/1:3, | | | | | |
| | 2.4% paraffin | | | | | |
| 574ROTTDS | SRS7-4501 | 2.90 | 3.11 | 1.95 | ./. | ./. |
| | 2% paraffin | (11% RSD) | (11% RSD) | (14% RSD) | | |
| 602ROTTDS | 7-4501:4601/ | 1.93 | 0.95 | 0.42 | ./. | ./. |
| | 1:3 | (4% RSD) | (25% RSD) | (27% RSD) | | |
| | 1% paraffin | | | | | |
| 611ROTTDS | 7-4501:4601/ | 3.33 | 1.28 | 0.95 | ./. | ./. |
| | 1:3 | (8% RSD) | (18% RSD) | (18% RSD) | | |
| | 2% paraffin | | | | | |
| 614ROTTDS | 7-4601 | 3.67 | 1.43 | ./. | ./. | ./. |
| | 2% paraffin | (6% RSD) | (8% RSD) | | | |
| 616ROTTDS | SRS7- | 4.63 | ./. | ./. | ./. | ./. |
| | 4501:4601/ | (6% RSD) | | | | |
| | 1:3 | | | | | |
| | 2.1% paraffin | | | | | |

### C) Tack

Finally, the tack of various TTS formulations comprising rotigotine and different non-amine-resistant silicone adhesives and adhesive mixtures has been compared with a placebo formulation and Neupro® available on the market after storage over 0 to 3 month(s).

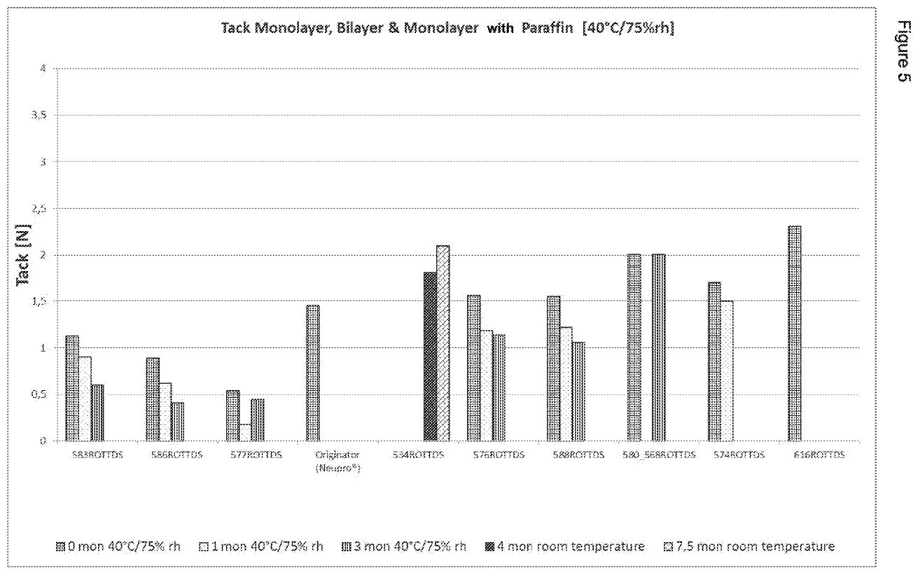

As can clearly be seen in Table 7 and FIG. 5 TTS formulations with rotigotine in combination with one or more non-silanol-reduced silicone adhesives (BIO-PSA 7-4501 and BIO-PSA 7-4601 by Dow Corning®) with still free silanol groups (non-silanol-reduced non-amine-resistant silicone adhesives) resulted in a lower tack than the Neupro® product.

Surprisingly, the tack when using silanol-reduced non-amine-resistant silicone adhesives was significantly higher, only slightly decreased during storage at 40° C./75% rh after 1 month and then, stabilized after 3 months. Moreover, said tack could be increased above the tack of the Neupro® product by adding a small amount of paraffin and surprisingly, the tack decreased less strongly during storage when using a small amount of paraffin (ca. 2% by weight).

TABLE 7
Tack of silicone adhesive formulations with non-silanol-reduced silicone adhesives
and with silanol-reduced silicone adhesives in different polymer adhesive mixing
ratios with or without paraffin when stored over 0-3 month(s) at 40° C./75% rh.

| | | | Tack after | Tack after |
|---|---|---|---|---|
| | | Tack after | 1 mon at | 3 mon at |
| Batch | Adhesive Type/Mixing Ratio | 0 mon | 40° C./75% rh | 40° C./75% rh |
| 583ROTTDS | 7-4601 | 1.13 | 0.90 | 0.60 |
| | | (5% RSD) | (11% RSD) | (28% RSD) |
| 586ROTTDS | 7-4501:4601/1:3 | 0.89 | 0.62 | 0.41 |
| | | (15% RSD) | (20% RSD) | (30% RSD) |
| 577ROTTDS | SRS7-4501 | 0.54 | 0.18 | 0.45 |
| | | (10% RSD) | (18% RSD) | (50% RSD) |
| Neupro ® lot: | | 1.45 | ./. | ./. |
| 55338204 | | (8% RSD) | | |
| 534ROTTDS | SRS7-4501:4601/1:1 | ./. | 2.02 | ./. |
| | | | (16% RSD) | |
| 576ROTTDS | SRS7-4601 | 1.71 | 1.18 | 1.14 |
| | | (9% RSD) | (2% RSD) | (11% RSD) |
| 588ROTTDS | SRS7-4501:4601/1:3 | 1.55 | 1.22 | 1.06 |
| | | (16% RSD) | (2% RSD) | (6% RSD) |
| 571_568ROTTDS | RS: SRS7-4501 | 2.01 | ./. | 2.01 |
| | HS: SRS7-4601 | (9% RSD) | | (4% RSD) |
| 592_591 ROTTDS | RS: SRS7-4501 | 2.06 | 1.41 | 0.97 |
| | HS: SRS7-4601 | (14% RSD) | (22% RSD) | (6% RSD) |
| 618_617ROTTDS | RS: SRS7-4501 | 2.25 | 1.53 | ./. |
| | HS: SRS7-4501:SRS7-4601/1:3, | (9% RSD) | (24% RSD) | |
| | 2.4% Paraffin | | | |
| 574ROTTDS | SRS7-4501 | 1.69 | 1.50 | ./. |
| | 2% Paraffin | (5% RSD) | (3% RSD) | |
| 599ROTTDS | SRS7-4501 | 0.80 | 0.62 | 0.39 |
| | 1% Paraffin | (28% RSD) | (12% RSD) | (16% RSD) |
| 611ROTTDS | 7-4501:4601 1:3 | 1.32 | 1.00 | 0.67 |
| | 2% Paraffin | (3% RSD) | (36% RSD) | (5% RSD) |
| 616ROTTDS | SRS7-4501:4601/1:3 | 2.31 | ./. | ./. |
| | 2.1% Paraffin | (1% RSD) | | |

### Example 3 (Recrystallization in Storage)

It was the aim to investigate the effect of the amount of PVP K90 on the recrystallization of rotigotine and permeation. When using a mixture of the non-amine-resistant silicone adhesives BIO-PSA SRS7-4501 and BIO-PSA SRS7-4601 (Dow Corning®) in the matrix layer together with rotigotine and PVP K90 crystallization was observed with weight ratios of rotigotine:PVP K90 in the range of from 9:3.2 to 9:5 after storage at 25° C. and 40° C.

Surprisingly, the use of the same mixture of the non-amine-resistant silicone adhesives BIO-PSA SRS7-4501 and BIO-PSA SRS7-4601 (Dow Corning®) in the matrix layer together with rotigotine and PVP K90 prevented crystallization at weight ratios of rotigotine:PVP K90 of 9:7 or less with the same storing temperatures.

TABLE 8
Recrystallization in Storage at 2-8° C., 25° C./60% rh or at 40° C./75% rh

| | | | | 511ROTTDS | | |
|---|---|---|---|---|---|---|
| | | | | or | | |
| Batch | 506ROTTDS | 525ROTTDS | 526ROTTDS | 538ROTTDS | 513ROTTDS | 554ROTTDS |
| Weight per | 50 | 50 | 50 | 50 | 50 | 50 |
| unit area | | | | | | |
| [g/m²] | | | | | | |
| rotigotine:PVP | 9:3.2 | 9:5 | 9:6 | 9:7 | 9:8 | 9:9 |
| K90 Ratio | | | | | | |
| [w:w] | | | | | | |
| Storage | 22 weeks | 12 months | 12 months | 15 months | 14.5 months | 5 months no |
| Period | crystals | no crystals | no crystals | no crystals | no crystals | crystals |
| Fridge | visible | visible | visible | visible | visible | visible |
| 2-8° C. | | | | | | |
| Storage | 6 weeks | 10 months | 10 months | 16 months | 14.5 months | 5 months no |
| Period | crystals | crystals | possibly | no crystals | no crystals | crystals |
| 25° C./60° rH | visible | visible | crystals | visible | visible | visible |
| | | | visible | | | |
| Storage | 3 weeks | 10 months | 10 months | 15 months | 14.5 months | 5 months no |
| Period | crystals | crystals | crystals | no crystals | no crystals | crystals |
| 40° C./75° rh | visible | visible | visible | visible | visible | visible |

### Example 4 (In Vitro Permeation and Dissolution)

### Description of the In Vitro Dissolution Experiments

Samples without a release liner of a certain size (e.g., 10 cm²) were exposed by means of the rotating cylinder according to Ph. Eur. 2.9.4 (Method 3) or USP <724> apparatus 6 (cylinder with adapter). 900 mL of 50 mM phosphate buffer (pH 4.5) were used as the exposing medium for each sample. The exposing temperature was 32° C., at a rotation speed of the cylinder of 50 rpm. Samplings were made depending on the formulation to be investigated at e.g., 0.25; 0.5; 0.75; 1; 1.5; 2; 2.5; and 3 hours (monolayer) and e.g., 0.5; 1; 1.5; 2; 2.5; 3; 4; and 6 hours (bilayer). Solutions of the samples were directly analyzed by RP-HPLC, as briefly described below:

Stationary phase: C18 (e.g., 50×3 mm, 5 μm particle size, 35° C. furnace temperature). Mobile phase: 70 mM phosphate buffer (pH 5.0)/methanol; 55/45 (v/v), with flow of 0.6 mL/min. Injection volume: 20 μL, with detection at 223 nm, retention time of rotigotine ca. 3-6 minutes, running time 8 minutes (isocratic). Evaluation was made via 1 point calibration by means of an external standard solution. Calculation of the cumulative release [%] was based on the average concentration in solutions of the samples.

### Description of the In Vitro Skin Permeation for Rotigotine

The investigations regarding the in vitro skin permeation were carried out with a skin permeation plant by NovoCell Schonbach according to OECD (2004) Test Guideline 428 “Skin absorption: In vitro Method & Series on testing and assessment”, No. 28 “Guidance document for the conduct of skin absorption studies”. A measuring cell was tempered to 32±1° C. throughout the measurement. The measuring cell consists of a donor and a acceptor chamber which are separated from each other by a heat separated epidermis of human skin with an effective permeation surface of 1.05 cm² which lies on the cellulose membrane. The matrix of the plaster to be tested (ca. 1.2 cm² in size) was adhered to the stratum corneum, the surface area to be exposed was facing the acceptor chamber. The measuring cell contained a total volume of 15 mL and was filled with physiological phosphate buffer, pH 5.5. Aliquotes have been taken as samples from the acceptor chamber at defined times (e.g., 1, 2, 3, 6, 9, 12, 15, 18, 21, and 24 hours), the concentration of rotigotine was determined via RP-HPLC analytics, and aliquot taken was immediately replaced by fresh buffer. Homogeneous distribution of the temperature and concentration of rotigotine was ensured by an integrated magnetic stirring system in the acceptor chamber.

Performing the RP-HPLC analytics and calculation of the sample concentrations were made as for the in vitro dissolution (see above). Thereafter, the cumulated permeated amount per time was calculated and plotted against time in a graph, and then the steady state flux was calculated [μg/cm²/h].

### A) Monolayer: Effect of the Ratios of Rotigotine to PVP K90 and Paraffin on Permeation

Skin permeation was investigated by applying the test formulations to human heat separated epidermis (HSE). Here, various weight ratios of rotigotine to PVP K90, with or without paraffin as an adhesion strength and tack increasing substance, have been tested.

Former monolayer formulation approaches with polyacrylate adhesives, mixtures of silicone adhesives with polyacrylates, polyisobutylene/polybutylene, styrene-butadiene or styrene-isoprene/resin formulations, respectively, as well as bilayer formulations using an initially active ingredient-free pressure sensitive adhesive layer with said adhesives without exception resulted in a relevantly lower permeation when applied to human heat separated epidermis (HSE) compared to Neupro®.

The effect of different rotigotine:PVP ratios with a fixed rotigotine content of 9% on the in vitro permeation was investigated when applying the monolayer test formulations to human HSE (heat separated epidermis).

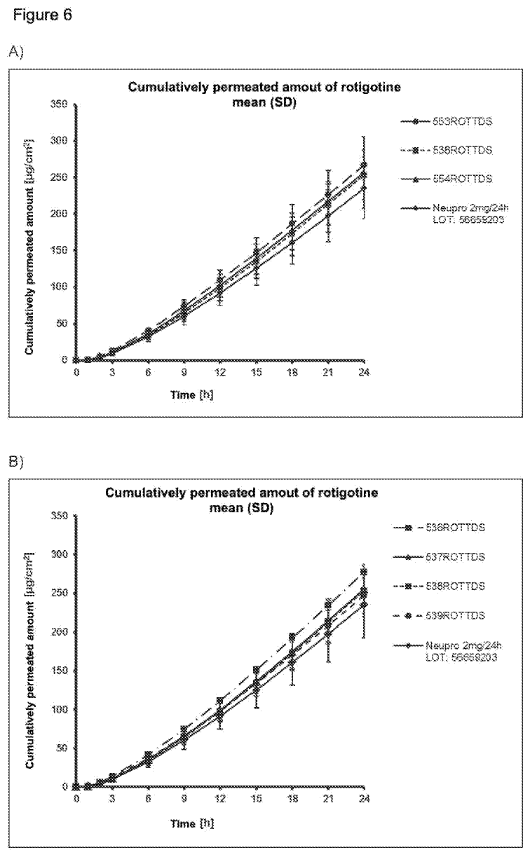

As seen in FIG. 6A), the amount of PVP K90 used in the range of from 6.4-9.0% with the rotigotine:PVP K90 ratios used had no relevant effect on the in vitro permeation. Moreover, the monolayer formulations showed an analogous, and even minimally higher/more effective permeation than Neupro®.

The effect of different mixing ratios of BIO-PSA SRS7-4501 to BIO-PSA SRS7-4601 with a fixed rotigotine content (9% by weight) and PVP K90 content (7% by weight) on the in vitro permeation was investigated when applying monolayer test formulations to human HSE (heat separated epidermis).

As seen in FIG. 6 B), in case of the monolayer formulations the mixing ratios of BIO-PSA SRS7-4501 to SRS7-4601 used of 1:1, 1:1.5, 1:2 and 1:3 had no relevant effect on the in vitro permeation when applying the test formulations to human HSE (heat separated epidermis). Also, the monolayer formulations with BIO-PSA SRS7-4501 to SRS7-4601 mixing ratios of 1:1 to 1:3 exhibited analogous and minimally higher/more effective permeation than Neupro®.

Finally, the effect of a small amount of paraffin on the in vitro permeation was investigated when applying monolayer test formulations to human HSE (heat separated epidermis).

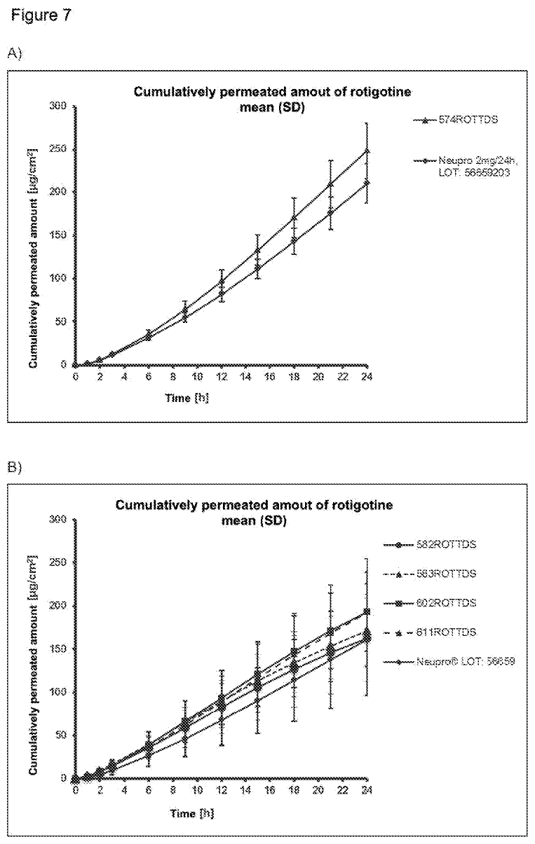

In case of the monolayer formulation with the silanol-reduced non-amine-resistant silicone adhesive BIO-PSA SRS7-4501, with an amount of paraffin of 2% by weight, a positive effect on the in vitro permeation has been shown, wherein the permeation was slightly increased/more effective compared to Neupro® (FIG. 7A).

In the case of monolayer formulations with non-silanol-reduced non-amine-resistant silicone adhesives BIO-PSA 7-4501 and/or BIO-PSA 7-4601 a positive effect on the in vitro permeation has been shown with an amount of paraffin of 1-2% by weight. Permeation was slightly higher/more effective compared with the Neupro® product. Also, the paraffin improved formulations exhibited a higher permeation than the formulations with these non-silanol-reduced non-amine-resistant silicone adhesives without paraffin (FIG. 7 B).

### B) Bilayer: Effect of Various Active Ingredient-Free Pressure Sensitive Adhesive Layers on Permeation

The effect of various initially active ingredient-free pressure sensitive adhesive layers based on different adhesive systems in combination with an active ingredient-containing silicone adhesive-based matrix layer (bilayer formulations) on the in vitro permeation was investigated when applied to human HSE (heat separated epidermis) compared to Neupro®.

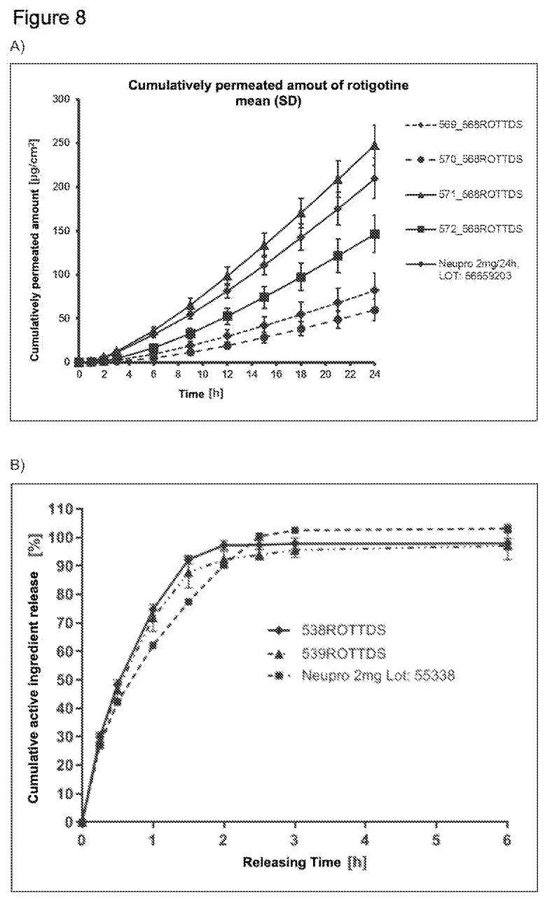

The use of initially active ingredient-free pressure sensitive adhesive layers coated to ca. 30 g/m² based on polyacrylate adhesive, polyisobutylene/polybutylene or a mixture of styrene isobutadiene, resin and paraffin were clearly inferior to the bilayer formulation based on an active ingredient-free pressure sensitive adhesive layer made of a non-amine-resistant silicone adhesive SRS7-4601 (cf., FIG. 8A). In all formulations the same matrix layer with 15% by weight of rotigotine, 8.5% by weight of PVP K90 and 76.5% by weight of BIO-PSA SRS7-4501 adhesive, coated to ca. 30 g/m², has been used. Also, the bilayer with a non-amine-resistant silicone adhesive in the initially active ingredient-free pressure sensitive adhesive layer and in the matrix layer exhibited an increased/enhanced permeation compared to Neupro®.

### C) In Vitro Dissolution

As shown in FIG. 8 B), the mixing ratios of BIO-PSA SRS7-4501 to SRS7-4601 used of 1:1 and 1:3 had no relevant effect on the in vitro release, since 538ROTTDS and 539ROTTDS behaved almost identically. However, the formulations with 9% by weight of rotigotine and 7% by weight of PVP K90 exhibited an initially slightly faster in vitro release than Neupro® what may be due to the higher content of PVP K90.

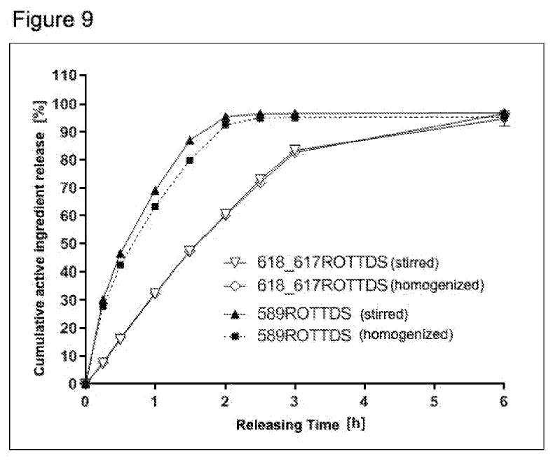

FIG. 9 shows the cumulatively released amount of rotigotine over 6 h, in case of a bilayer formulation via in vitro dissolution, in which the mass of the active ingredient-containing matrix layer (2) before the respective coating to ca. 50 g/m² each was only stirred and the same mass before the respective coating to 50 g/m² was additionally homogenized. Then, both active ingredient-containing matrix layers (2) were laminated with the same initially active ingredient-free adhesive layer (3). As a further comparison a monolayer formulation was treated in analogy, wherein no initially active ingredient-free adhesive layer (3) was laminated. Only slow stirring resulted in larger spheres with a wider size distribution compared to later additional homogenizing. Both showed an identic course of release.

1. A transdermal therapeutic system comprising:
(i) a backing layer (1),
(ii) a matrix layer (2) containing a drug, wherein the drug is rotigotine; and
(iii) a release liner (4) to be removed before use,
wherein the matrix layer (2) contains one or more non-amine-resistant pressure sensitive silicone adhesives in an amount of more than 50% by weight, based on the total weight of the pressure sensitive adhesives of the matrix layer (2), and paraffin in an amount of at least 0.1% by weight, based on the total weight of the matrix layer (2).

2. The transdermal therapeutic system according to claim 1, wherein the matrix layer (2) contains paraffin in an amount of 0.2-20.0% by weight, based on the total weight of the matrix layer (2).

3. The transdermal therapeutic system according to claim 1, wherein the matrix layer (2) has a weight per unit area of 40-70 g/m².

4. The transdermal therapeutic system according to claim 1, wherein at least one additional, initially active ingredient-free, pressure sensitive adhesive layer (3) is between the matrix layer (2) and the release liner (4) to be removed before use, further wherein the at least one additional initially active ingredient-free pressure sensitive adhesive layer (3) contains one or more non-amine-resistant pressure sensitive silicone adhesives in an amount of more than 50% by weight, based on the total weight of the pressure sensitive adhesives of the at least one additional initially active ingredient-free pressure sensitive adhesive layer (3), and paraffin in an amount of at least 0.1% by weight based on the total weight of the at least one additional initially active ingredient-free pressure sensitive adhesive layer (3).

5. The transdermal therapeutic system according to claim 4, wherein the at least one additional initially active ingredient-free pressure sensitive adhesive layer (3) has a weight per unit area of 20-40 g/m².

6. The transdermal therapeutic system according to claim 1, wherein the matrix layer (2) has a weight percentage of non-amine-resistant pressure sensitive silicone adhesive of more than 75% by weight, based on the total weight of the pressure sensitive adhesive of the matrix layer (2).

7. The transdermal therapeutic system according to claim 4, wherein the at least one additional, initially active ingredient-free, pressure sensitive adhesive layer (3) has a weight percentage of non-amine-resistant pressure sensitive silicone adhesive of more than 75% by weight, based on the total weight of the pressure sensitive adhesive of the at least one additional initially active ingredient-free pressure sensitive adhesive layer (3).

8. The transdermal therapeutic system according to claim 1, wherein the matrix layer (2) and, if present, the at least one additional, initially active ingredient-free, pressure sensitive adhesive layer (3), exclusively have non-amine-resistant pressure sensitive silicone adhesives as the pressure sensitive adhesives.

9. The transdermal therapeutic system according to claim 1, wherein the non-amine-resistant pressure sensitive silicone adhesives are silanol-reduced silicone adhesives.

10. The transdermal therapeutic system according to claim 1, wherein the rotigotine in the matrix layer (2) is substantially present in a non-crystalline form in the dispersed phase of a solid dispersion comprising a polyvinylpyrrolidone.

11. The transdermal therapeutic system according to claim 10, wherein the weight ratio of rotigotine to polyvinylpyrrolidone is at most 9:6.4.

12. The transdermal therapeutic system according to claim 11, wherein the weight ratio of rotigotine to polyvinylpyrrolidone is 9:7 to 9:10.

13. The transdermal therapeutic system according to claim 1, wherein the content of active ingredient in the matrix layer (2) is in the range of from 6% by weight to 20% by weight, based on the total weight of the matrix layer (2).

14. The transdermal therapeutic system according to claim 1, wherein the one or more non-amine-resistant, pressure sensitive silicone adhesives of the matrix layer (2) and/or the at least one additional, initially active ingredient-free, pressure sensitive adhesive layer (3), if said additional layer (3) is present, comprise two or more non-amine-resistant pressure sensitive silicone adhesives, further wherein preferably one or more of the non-amine-resistant pressure sensitive silicone adhesives have a medium tack and one or more of the non-amine-resistant pressure sensitive silicone adhesives have a high tack.

15. The transdermal therapeutic system according to claim 1, wherein the one or more non-amine-resistant, pressure sensitive silicone adhesives of the matrix layer (2) and/or the at least one additional, initially active ingredient-free, pressure sensitive adhesive layer (3), if said additional layer (3) is present, exclusively consist of a silanol-reduced non-amine-resistant pressure sensitive silicone adhesive.

16. The transdermal therapeutic system according to claim 1, wherein the matrix layer (2) contains one or more antioxidants and/or sodium metabisulfite.

17. A method for treating Parkinson's disease in a patient diagnosed therewith, said method comprising the step of applying the transdermal therapeutic system according to claim 1 to the skin of said patient.

18. A method for the preparation of a transdermal therapeutic system according to claim 1 as a monolayer formulation, said method comprising the steps of:
(i) preparing a homogenized coating mass by adding together all components of the matrix layer (2) in a suitable solvent and mixing to the desired homogeneity;
(ii) applying the homogenized coating mass onto a backing layer (1) or a release liner (4) and removing the solvent by drying; and
(iii) laminating the release liner (4) or backing layer (1), onto the matrix layer (2), and blanking transdermal therapeutic systems of the suitable size.

19. The method for the preparation of a transdermal therapeutic system according to claim 1, said method comprising the steps of:
(i) preparing a first precursor of the transdermal therapeutic system by:
a1) preparing a first homogenized coating mass by adding together all the components of the matrix layer (2) in a suitable solvent and mixing to the desired homogeneity;
a2) applying the first homogenized coating mass onto a backing layer (1) or a temporary release liner (4) and removing the solvent by drying; and
a3) laminating the temporary release liner (4) or backing layer (1), onto the matrix layer (2);
(ii) preparing a second precursor of the transdermal therapeutic system by:
b1) preparing a homogenized coating mass by adding together all the components of the at least one additional, initially active ingredient-free, pressure sensitive adhesive layer (3) in a suitable solvent and mixing to the desired homogeneity;
b2) applying the further homogenized coating mass onto a release liner (4) and removing the solvent by drying; and
(iii) removing the temporary release liner (4) from the first precursor of the transdermal therapeutic system of a), laminating the first precursor and the second precursor of the transdermal therapeutic system to an overall laminate comprising in this layering order a backing layer (1), an active ingredient-containing matrix layer (2), at least one additional initially active ingredient-free, pressure sensitive adhesive layer (3) and a release liner (4), and blanking transdermal therapeutic systems of the suitable size.

20. The transdermal therapeutic system according to claim 1, wherein:
(i) the matrix layer (2) contains paraffin in an amount of 1.0-4.0% by weight, based on the total weight of the matrix layer (2);
(ii) the matrix layer (2) has a weight per unit area of 50-60 g/m;
(iii) the matrix layer (2) has a weight percentage of non-amine-resistant, pressure sensitive, silicone adhesive of more than 90% by weight, based on the total weight of the pressure sensitive adhesive of the matrix layer (2);
(iv) the weight ratio of rotigotine to polyvinylpyrrolidone is at most 9:7; and
(v) the content of active ingredient in the matrix layer (2) is in the range of from 6.5% by weight to 11.5% by weight, based on the total weight of the matrix layer (2).

21. The transdermal therapeutic system according to claim 4, wherein:
(i) the matrix layer (2) contains paraffin in an amount of 1.0-4.0% by weight, based on the total weight of the matrix layer (2);
(ii) the matrix layer (2) has a weight per unit area of 50-60 g/m;
(iii) the at least one additional, initially active ingredient-free, pressure sensitive adhesive layer (3) has a weight per unit area of 25-35 g/m²;
(iv) the matrix layer (2) has a weight percentage of non-amine-resistant, pressure sensitive, silicone adhesive of more than 90% by weight, based on the total weight of the pressure sensitive adhesive of the matrix layer (2);
(v) the weight ratio of rotigotine to polyvinylpyrrolidone is at most 9:7; and
(vi) the content of active ingredient in the matrix layer (2) is in the range of from 6.5% by weight to 11.5% by weight, based on the total weight of the matrix layer (2).
